(19) United States
(12) Patent Application Publication
(10) Pub. No.: US 2022/0144943 A1
(43) Pub. Date: May 12, 2022

(54) ANTI-CD47 ANTIBODIES AND USES THEREOF

(71) Applicant: INTEGRITY BIOVENTURES, INC., San Diego, CA (US)

(72) Inventors: Jason KAHANA, San Diego, CA (US); John LIPPINCOTT, San Diego, CA (US)

(21) Appl. No.: 17/429,331

(22) PCT Filed: Feb. 7, 2020
(86) PCT No.: PCT/US20/17221

§ 371 Date: Aug. 7, 2021

Related U.S. Application Data
(60) Provisional application No. 62/803,315, filed on Feb. 8, 2019.

(51) Int. Cl. C07K 16/28

(52) U.S. Cl. CPC C07K 16/2803; C07K 2317/24; C07K 2317/92; C07K 2317/76; C07K 2317/565

(57) ABSTRACT

Disclosed herein, in certain embodiments, are anti-CD47 antibodies, nucleic acid polymers encoding the antibodies, and vectors for expressing the same. In some embodiments, also disclosed herein include pharmaceutical compositions comprising the anti-CD47 antibodies and methods of treatment.

### CROSS REFERENCE

This application claims the benefit of U.S. Provisional Application No. 62/803,315 filed Feb. 8, 2019, which is incorporated by reference herein in its entirety.

### BACKGROUND OF THE DISCLOSURE

The immune system involves an interplay between the innate and adaptive defenses. The macrophages and neutrophils of the innate immune system provides a first line defense against microorganisms and foreign molecules while the lymphocytes of the adaptive immune system provide a second layer of protection. In some instances, diseases such as cancer and pathogens have evolved to by-pass these multi-layer defense mechanisms or have evolved protective mechanisms to evade such immune surveillance.

### SUMMARY OF THE DISCLOSURE

Disclosed herein, in certain embodiments, are anti-CD47 antibodies, nucleic acid polymers encoding the antibodies, and vectors for expressing the same. In some embodiments, also disclosed herein include pharmaceutical compositions comprising the anti-CD47 antibodies and kits. In additional embodiments, disclosed herein include methods of inducing phagocytosis and methods of treatment utilizing the anti-CD47 antibodies.

Disclosed herein, in certain embodiments, is an anti-CD47 antibody comprising a variable heavy chain (VH) region and a variable light chain (VL) region, wherein the VH region comprises: CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence IX₈X₉X₁₀X₁₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y. In some embodiments, the VH region comprises: CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence IX₈X₉X₁₀X₁₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence selected from SEQ ID NOs: 3-5, 7, 10, 13, 14, 17, 20, 22, 24, 27, 32, 35, 38, 41, and 44. In some embodiments, the VH region comprises: CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence selected from SEQ ID NOs: 2, 9, 12, 16, 19, 21, 26, 28, 29, 31, 34, 37, 40, and 43; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y. In some embodiments, the VH region comprises: CDR1 sequence selected from SEQ ID NOs: 1, 6, 8, 11, 15, 18, 23, 25, 30, 33, 36, 39, and 42; CDR2 sequence IX₈X₉X₁₀X₁₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y. In some embodiments, the VH region comprises: CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence selected from SEQ ID NOs: 2, 9, 12, 16, 19, 21, 26, 28, 29, 31, 34, 37, 40, and 43; and CDR3 sequence selected from SEQ ID NOs: 3-5, 7, 10, 13, 14, 17, 20, 22, 24, 27, 32, 35, 38, 41, and 44. In some embodiments, the VH region comprises: CDR1 sequence selected from SEQ ID NOs: 1, 6, 8, 11, 15, 18, 23, 25, 30, 33, 36, 39, and 42; CDR2 sequence selected from SEQ ID NOs: 2, 9, 12, 16, 19, 21, 26, 28, 29, 31, 34, 37, 40, and 43; and CDR3 sequence selected from SEQ ID NOs: 3-5, 7, 10, 13, 14, 17, 20, 22, 24, 27, 32, 35, 38, 41, and 44. In some embodiments, the VL region comprises: CDR1 sequence selected from SEQ ID NOs: 45, 48, 51, 55, 56, 61, 64, 67, 69, 71, or 74; CDR2 sequence selected from SEQ ID NOs: 46, 49, 52, 57, 62, 65, 72, or 75; and CDR3 sequence selected from SEQ ID NOs: 47, 50, 53, 54, 58, 59, 60, 63, 66, 68, 70, 73, or 76. In some embodiments, the VH region comprises CDR1 sequence selected from SEQ ID NOs: 1, 15, 36, and 39; CDR2 sequence selected from SEQ ID NOs: 2, 16, 37, and 40; and CDR3 sequence selected from SEQ ID NOs: 3, 4, 5, 17, 38, and 41; and the VL region comprises CDR1, CDR2, and CDR3 sequences of SEQ ID NOs: 45-47. In some embodiments, the VH region comprises CDR1, CDR2, and CDR3 sequences of SEQ ID NOs: 6, 2, and 7; and the VL region comprise CDR1, CDR2, and CDR3 sequences of SEQ ID NOs: 48-50. In some embodiments, the VH region comprises CDR1, CDR2, and CDR3 sequences of SEQ ID NOs: 23, 21, and 24; and the VL region comprise CDR1, CDR2, and CDR3 sequences of SEQ ID NOs: 51-53. In some embodiments, the VH region comprises CDR1, CDR2, and CDR3 sequences of SEQ ID NOs: 18, 21, and 22; and the VL region comprise CDR1, CDR2, and CDR3 sequences of SEQ ID NOs: 51, 52, and 54. In some embodiments, the VH region comprises CDR1, CDR2, and CDR3 sequences of SEQ ID NOs: 18-20; and the VL region comprise CDR1, CDR2, and CDR3 sequences of SEQ ID NOs: 55, 52, and 53. In some embodiments, the VH region comprises CDR1 sequence selected from SEQ ID NO: 25, CDR2 sequence selected from SEQ ID NOs: 26 and 28, and CDR3 sequence selected from SEQ ID NO: 27, and the VL region comprise CDR1, CDR2, and CDR3 sequences of SEQ ID NOs: 56-58. In some embodiments, the VH region comprises CDR1, CDR2, and CDR3 sequences of SEQ ID NOs: 25, 28, and 27; and the VL region comprise CDR1, CDR2, and CDR3 sequences of SEQ ID NOs: 56, 57, and 59. In some embodiments, the VH region comprises CDR1, CDR2, and CDR3 sequences of SEQ ID NOs: 25, 29, and 27; and the VL region comprise CDR1, CDR2, and CDR3 sequences of SEQ ID NOs: 56, 57, and 60. In some embodiments, the VH region comprises CDR1, CDR2, and CDR3 sequences of SEQ ID NOs: 30-32; and the VL region comprise CDR1, CDR2, and CDR3 sequences of SEQ ID NOs: 61-63. In some embodiments, the VH region comprises CDR1 sequence selected from SEQ ID NO: 11, CDR2 sequence selected from SEQ ID NO: 12, and CDR3 sequence selected from SEQ ID NOs: 13 and 14, and the VL region comprise CDR1, CDR2, and CDR3 sequences of SEQ ID NOs: 64-66. In some embodiments, the VH region comprises CDR1, CDR2, and CDR3 sequences of SEQ ID NOs: 8-10; and the VL region comprise CDR1, CDR2, and CDR3 sequences of SEQ ID NOs: 67, 65, and 68. In some embodiments, the VH region comprises CDR1, CDR2, and CDR3 sequences of SEQ ID NOs: 42-44; and the VL region comprise CDR1, CDR2, and CDR3 sequences of SEQ ID NOs: 69, 52, and 70. In some embodiments, the VH region comprises CDR1, CDR2, and CDR3 sequences of SEQ ID NOs: 33-35; and the VL region comprise CDR1, CDR2, and CDR3 sequences of SEQ ID NOs: 71-73. In some embodiments, the VH region comprises CDR1, CDR2, and CDR3 sequences of SEQ ID NOs: 11-13; and the VL region comprise CDR1, CDR2, and CDR3 sequences of SEQ ID NOs: 74-76. In some embodiments, the VH region comprises a sequence selected from Table 3. In some embodiments, the VL region comprises a sequence selected from Table 3. In some embodiments, the antibody is a full-length antibody. In some embodiments, the antibody is a binding fragment. In some embodiments, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the antibody comprises a humanized antibody or binding fragment thereof, a chimeric antibody or binding fragment thereof, a monoclonal antibody or binding fragment thereof, or a bispecific antibody or binding fragment thereof. In some embodiments, the antibody comprises an IgG1 framework sequence. In some embodiments, the antibody comprises an IgG2 framework sequence. In some embodiments, the antibody comprises an IgG4 framework sequence. In some embodiments, the antibody further comprises a mutation in the Fc region. In some embodiments, the mutation is S228P. In some embodiments, the antibody comprises a HC sequence selected from SEQ ID NO: 123-125. In some embodiments, the antibody comprises a LC sequence selected from SEQ ID NO: 126-128. In some embodiments, the antibody further comprises a payload. In some embodiments, the payload comprises a small molecule, a peptide, or a protein. In some embodiments, the antibody has a humanization score of greater than 85 in both the HC and LC sequences. In some embodiments, the antibody increases phagocytosis in a target cell relative to the phagocytic activity by a control antibody to an equivalent target cell. In some embodiments, the antibody has a decreased hemagglutination potential compared to the control antibody. In some embodiments, the decrease in hemagglutination potential is about 10%, 20%, 30%, 40%, 50%, 60%, 70%, 80%, 90%, 95%, 99%, or higher. In some embodiments, the decrease in hemagglutination potential is about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or more. In some embodiments, the antibody does not induce hemagglutination. In some embodiments, the control antibody is CC-90002 or hu5F9.

Disclosed herein, in certain embodiments, is a nucleic acid polymer encoding an anti-CD47 antibody described herein.

Disclosed herein, in certain embodiments, is a vector comprising a nucleic acid polymer encoding an anti-CD47 antibody described herein.

Disclosed herein, in certain embodiments, is a pharmaceutical composition comprising an anti-CD47 antibody described herein; and a pharmaceutically acceptable excipient. In some embodiments, the pharmaceutical composition is formulated for systemic administration. In some embodiments, the pharmaceutical composition is formulated for parenteral administration.

Disclosed herein, in certain embodiments, is a method of inducing phagocytosis of a target cell expressing CD47, comprising: contacting the target cell with an anti-CD47 antibody described herein for a time sufficient for binding of the antibody to CD47, wherein the anti-CD47 antibody blocks interaction of CD47 with SIRPPα expressed on a macrophage, thereby inducing phagocytosis of the target cell. In some embodiments, the target cell is a cancer cell. In some embodiments, the cancer cell is from bladder cancer, bone cancer, brain cancer, breast cancer, colorectal cancer, eye cancer, head and neck cancer, kidney cancer, liver cancer, lung cancer, pancreatic cancer, prostate cancer, skin cancer, stomach cancer, thyroid cancer, or uterine cancer. In some embodiments, the cancer cell is from chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the target cell is a pathogen-infected cell. In some embodiments, the pathogen is a virus, a bacterium, or a protozoan. In some embodiments, the method is an in vivo method. In some embodiments, the method is an in vitro or ex vivo method.

Disclosed herein, in certain embodiments, is a method of treating a disease or condition in a subject in need thereof, comprising: administering to the subject an anti-CD47 antibody described herein or a pharmaceutical composition described herein, thereby treating the disease or condition in the subject. In some embodiments, the subject has a cancer. In some embodiments, the cancer is a solid tumor. In some embodiments, the solid tumor is bladder cancer, bone cancer, brain cancer, breast cancer, colorectal cancer, eye cancer, head and neck cancer, kidney cancer, liver cancer, lung cancer, ovarian cancer, pancreatic cancer, prostate cancer, skin cancer, stomach cancer, thyroid cancer, or uterine cancer. In some embodiments, the cancer is a hematologic malignancy. In some embodiments, the hematologic malignancy is a B cell lymphoma. In some embodiments, the hematologic malignancy is a T cell lymphoma. In some embodiments, the hematologic malignancy is a Hodgkin's lymphoma. In some embodiments, the hematologic malignancy is a non-Hodgkin's lymphoma. In some embodiments, the hematologic malignancy is chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the cancer is a metastatic cancer. In some embodiments, the cancer is a relapsed or refractory cancer. In some embodiments, the subject has a pathogenic infection. In some embodiments, the method further comprises administering an additional therapeutic agent. In some embodiments, the additional therapeutic agent comprises an immune checkpoint modulator. In some embodiments, the immune checkpoint modulator is a modulator of PD-1, PD-L1, PD-L2, CTLA-4, TIM3, LAG3, B7-H3, KIR, 4-1BB, GITR, PS, CD52, CD30, CD20, CD33, CD27, OX40, ICOS, BTLA (CD272), CD160, 2B4, LAIR1, TIGHT, LIGHT, DR3, CD226, CD2, or SLAM. In some embodiments, the immune checkpoint inhibitor is Pembrolizumab, Nivolumab, Atezolizumab, Avelumab, Durvalumab, or Ipilimumab. In some embodiments, the additional therapeutic agent comprises chemotherapeutic agent, immunotherapeutic agent, targeted therapeutic agent, hormone-based therapeutic agent, stem-cell based therapeutic agent, or radiation. In some embodiments, the additional therapeutic agent comprises a first-line therapeutic agent. In some embodiments, the additional therapeutic agent and the antibody are administered simultaneously. In some embodiments, the additional therapeutic agent and the antibody are administered sequentially. In some embodiments, the additional therapeutic agent is administered prior to the antibody. In some embodiments, the additional therapeutic agent is administered after administration of the antibody. In some embodiments, the additional therapeutic agent and the antibody are administered as a separate dosage. In some embodiments, the subject has undergone surgery. In some embodiments, the subject is a human.

Disclosed herein, in certain embodiments, is a kit comprising an anti-CD47 antibody described herein, a vector described herein, or a pharmaceutical composition described herein.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings below. The patent application file contains at least one drawing executed in color. Copies of this patent application publication with color drawing(s) will be provided by the Office upon request and payment of the necessary fee.

FIG. 1A-FIG. 1B illustrate exemplary bispecific antibody formats contemplated herein. FIG. 1A and FIG. 1B are adapted from FIG. 2 of Brinkmann and Kontermann, “The making of bispecific antibodies,” MABS 9(2): 182-212 (2017).

FIG. 2 shows the percentage if inhibition in comparison with a reference antibody 5F9 (Forty Seven, Inc.).

FIG. 3 shows the mean fluorescence intensity (MFI) of 10-17I-A in comparison to a reference antibody 5F9.

FIG. 4 shows the pyagocytosis of Raji cells in the presence of an IgG1 control antibody, 10-17I-A, and reference antibody 5F9.

FIG. 5 shows a lack of hemagglutination of exemplary anti-CD47 antibodies described herein and reference antibody 5F9.

### DETAILED DESCRIPTION OF THE DISCLOSURE

Cluster of differentiation 47 (CD47), also known as integrin-associated protein (IAP), is an ubiquitously expressed membrane protein of the immunoglobulin superfamily with a single IgV-like domain at its N-terminus, a hydrophobic stretch with five membrane-spanning segments and an alternatively spliced cytoplasmic C-terminus. CD47 was originally identified as a membrane protein involved in β3 integrin-mediated signaling on leukocytes, but has since been identified to also interact with signal regulatory protein-alpha (SIRPα), thrombospondin-1, and additional proteins that regulate cell migration, axon extension, cytokine production, and T cell activation.

SIRPα, also known as Src homology 2 domain-containing protein tyrosine phosphatase substrate 1/brain Ig-like molecule with tyrosine-based activation motif/cluster of differentiation antigen-like molecule with tyrosine-based activation motif/cluster of differentiation antigen-like family member A (SHPS-1/BIT/CD172), is also a member of the immunoglobulin superfamily and is abundantly expressed in myeloid-lineage hematopoietic cells such as macrophages and dendritic cells. Interaction of CD47 with SIRPPα results in phosphorylation of the cytoplasmic immunoreceptor tyrosine-based inhibition (ITIM) motif on SIRPα, leading to recruitment of SUP-1 and SHP-2 phosphatases. The subsequent downstream effect is inhibition of myosin-IIA accumulation at the phagocytic synapse and thereby the inhibition of phagocytosis. As such, CD47 is considered as a negative immune checkpoint that sends a “don't eat me” signal by cells to prevent phagocytosis.

In some instances, most types of tumor cells ubiquitously overexpress CD47. Indeed, a study has shown that cancer stem cells (CSCs), which have been implicated in cancer recurrence, increases CD47 expression to protect themselves from immune-mediated elimination during conventional anti-tumor therapies (Soltanian S, Matin M M. “Cancer stem cells and cancer therapy,” Tumour Biol. 32(3):425-440 (2011)).

Programmed cell death and phagocytic cell removal are different methods utilized by damaged or infected cells responding to pathogenic threats to the organism. In some instances, pathogens also utilize a CD47-mediated process to evade immune clearance by phagocytic cells.

Thrombospondin-1 (TSP-1) is a member of the thrombospondin (TSP) family of secreted glycoproteins. The TSP family comprises five members that modulate a variety of cellular functions including angiogenesis, apoptosis, synaptogenesis, platelet aggregation, inflammatory response, wound repair, and fibrosis. TSP-1 is a 420-450 kDa trimeric glycoprotein that is expressed by platelets, endothelial cells, smooth-muscle cells, leukocytes, and activated astrocytes. Activation of CD47 by TSP-1 facilitates the generation of CD4+ Foxp3+ T regulatory (Treg) cells. The presence of CD4+ Foxp3+ Treg cells lead to suppression of the proliferation and cytokine production of autologous Tho, Th1, and Th2 cells via a contact-dependent mechanism.

In some embodiments, disclosed herein are anti-CD47 antibodies for use in the treatment of a proliferative disease, an inflammatory disease, and/or a pathogenic infection. In some instances, the anti-CD47 antibodies described herein are humanized antibodies with decreased levels of hemagglutination compared to a reference antibody (e.g., a commercially available anti-CD47 antibody). In some cases, the anti-CD47 antibodies described herein have improved humanization scores compared to a reference antibody.

### Anti-CD47 Antibodies

Disclosed herein, in certain embodiments, is an anti-CD47 antibody comprising a variable heavy chain (VH) region and a variable light chain (VL) region, in which the VH region comprises CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence IX₈X₉X₁₀X₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

In some embodiments, the VH region comprises CDR1 sequence GFNIKDX₆Y, in which X₆ is selected from Y, H, or T; CDR2 sequence IDPX₁₀NGX₁₃T, in which X₁₀ is selected from E or A, and X₁₃ is selected from D or N; CDR3 sequence X₁₄X₁₅X₁₆X₁₇X₁₈X₁₉X₂X₂₁X₂₂X₂₃X₂₄X₂₅X₂₆, X₁₄ is selected from N or G; X₁₅ is selected from A or R; X₁₆ is selected from W or G; X₁₇ is selected from G, L, or D; X₁₈ is selected from G, E, F, or Y; X₁₉ is selected from S, N, R, D, or Y; X₂₀ is selected from S, Y, or A; X₂₁ is either present or absence, if present, is selected from S, P, or M; X₂₂ is either present or absence, if present, is selected from A or D; X₂₃ is either present or absence, if present, is selected from W or Y; X₂₄ is either present or absence, if present, is F; X₂₅ is either present or absence, if present, is A; and X₂₆ is either present or absence, if present, is Y.

In some embodiments, the VH region comprises CDR1 sequence GYTFX₄X₅X₆X₇, in which X₄ is selected from T or S; X₅ is selected from D, E, or S; X₆ is selected from Y, N, or H; X₇ is selected from W, T, or I; CDR2 sequence IX₈X₉X₁₀X₁₁X₁₂X₁₃T, in which X₈ is selected from D, S, N, or L; X₉ is selected from P, C, or T; X₁₀ is selected from Y, S, N, or G; X₁₁ is selected from N, D, I or S; X₁₂ is selected from G, S, C, or T; X₁₃ is selected from A, Y, G, E, or S; and CDR3 sequence X₁₄RX₁₆X₁₇X₁₈X₁₉X₂₀X₂₁X₂₂X₂₃X₂₄X₂₅, in which X₁₄ is selected from G or A; X₁₆ is selected from G, R, Y, or L; X₁₇ is selected from D, A, S, Y, or G; X₁₈ is selected from F, Y, R, L, or T; X₁₉ is selected from Y, A, F, L, or G; X₂₀ is selected from A, M, D, W, R, or V; X₂₁ is selected from M, N, Y, G, F, or A; X₂₂ is either present or absence, if present, is selected from D, Y, A, or T; X₂₃ is either present or absence, if present, is selected from Y, F, V, or D; X₂₄ is either present or absence, if present, is selected from D or Y; and X₂₅ is either present or absence, if present, is Y.

In some embodiments, the VH region comprises CDR1, CDR2, and CDR3 sequences selected from Table 1.

| | | SEQ | | SEQ | | SEQ ID |
|---|---|---|---|---|---|---|
| NAME | VH_CDR1 | ID NO: | VH_CDR2 | ID NO: | VH_CDR3 | NO: |
| IBV2039 | GFNIKDYY | 1 | IDPENGDT | 2 | NAWGGSSSAWFAY | 3 |
| IBV2041 | GFNIKDYY | 1 | IDPENGDT | 2 | NAWGGNYPAWFAY | 4 |
| IBV2043 | GFNIKDYY | 1 | IDPENGDT | 2 | NAWLERAMDY | 5 |
| IBV2042 | GFNIKDHY | 6 | IDPENGDT | 2 | NAGLFDY | 7 |
| IBV2058 | GFNIKDTY | 8 | IDPANGNT | 9 | GRGDYYAMDY | 10 |
| IBV2101* | GYSFTGYY | 11 | ISCYNGAT | 12 | ARRDFYGMDY | 13 |
| IBV2052 | GYSFTGYY | 11 | ISCYNGAT | 12 | ARRDFYAMDY | 14 |
| IBV2044 | GYTFTDYW | 15 | IDTSDSYT | 16 | ARGDYAMNY | 17 |
| IBV2051 | GYTFTEYT | 18 | INPNIGAT | 19 | ARRAYFDY | 20 |
| IBV2047 | GYTFTEYT | 18 | INPNNGGT | 21 | GRRSYFDY | 22 |
| IBV2046 | GYTFTEYI | 23 | INPNNGGT | 21 | ARRAYFDY | 24 |
| IBV2048 | GYTFTSNW | 25 | IDPYDCET | 26 | ARYYRYDGYFDY | 27 |
| IBV2040 | GYTFTSNW | 25 | IDPYDSET | 28 | ARYYRYDGYFDY | 27 |
| IBV2050 | GYTFTSNW | 25 | IDPYDTET | 29 | ARYYRYDGYFDY | 27 |
| IBV2053 | GYTFTSNW | 25 | IDPYDSET | 28 | ARYYRYDGYFDY | 27 |
| IBV2054 | GYTFTSHW | 30 | IDPSDSET | 31 | ARLGRAWFAY | 32 |
| IBV2056 | GYTFSSYW | 33 | ILPGSGST | 34 | ARRGLLRFDV | 35 |
| IBV2057 | GYTFTDYW | 36 | IDTSDSYT | 37 | ARLGTGVATDY | 38 |
| IBV2045 | GHTFTDYW | 39 | IDNSDSYT | 40 | AKGYYAMDY | 41 |
| IBV_H1 | GYAFTNYL | 42 | INPGSGAT | 43 | ARRVYFDY | 44 |

*IBV2101 shares the same VH CDR1, CDR2, and CDR3 sequences with IBV2049

In some embodiments, the VH region comprises CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence IX₈X₉X₁₀X₁₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence selected from SEQ ID NOs: 3-5, 7, 10, 13, 14, 17, 20, 22, 24, 27, 32, 35, 38, 41, and 44.

In some embodiments, the VH region comprises CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence selected from SEQ ID NOs: 2, 9, 12, 16, 19, 21, 26, 28, 29, 31, 34, 37, 40, and 43; and CDR3 sequence X₁₄X₁₅X₁₆X₁₇X₁₈X₁₉X₂X₂X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

In some embodiments, the VH region comprises CDR1 sequence selected from SEQ ID NOs: 1, 6, 8, 11, 15, 18, 23, 25, 30, 33, 36, 39, and 42; CDR2 sequence IX₈X₉X₁₀X₁₁X₁₂X₁₃T, wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

In some embodiments, the VH region comprises CDR1 sequence selected from SEQ ID NOs: 1, 6, 8, 11, 15, 18, 23, 25, 30, 33, 36, 39, and 42; CDR2 sequence selected from SEQ ID NOs: 2, 9, 12, 16, 19, 21, 26, 28, 29, 31, 34, 37, 40, and 43; and CDR3 sequence X₁₄X₁₅X₁₆X₁₇X₁₈X₁₉X₂₀X₂₁X₂₂X₂₃X₂₄X₂₅X₂₆, wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

In some instances, the VH region comprises CDR1 sequence GX₁X₂X₃X₄X₅X₆X₇, wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L; CDR2 sequence selected from SEQ ID NOs: 2, 9, 12, 16, 19, 21, 26, 28, 29, 31, 34, 37, 40, and 43; and CDR3 sequence selected from SEQ ID NOs: 3-5, 7, 10, 13, 14, 17, 20, 22, 24, 27, 32, 35, 38, 41, and 44.

In some instances, the VH region comprises CDR1 sequence selected from SEQ ID NOs: 1, 6, 8, 11, 15, 18, 23, 25, 30, 33, 36, 39, and 42; CDR2 sequence selected from SEQ ID NOs: 2, 9, 12, 16, 19, 21, 26, 28, 29, 31, 34, 37, 40, and 43; and CDR3 sequence selected from SEQ ID NOs: 3-5, 7, 10, 13, 14, 17, 20, 22, 24, 27, 32, 35, 38, 41, and 44.

In some embodiments, the VL region comprises CDR1, CDR2, and CDR3 sequences selected from Table 2.

| | | SEQ ID | | SEQ ID | | SEQ ID |
|---|---|---|---|---|---|---|
| NAME | VL_CDR1 | NO: | VL_CDR2 | NO: | VL_CDR3 | NO: |
| IBV2039¹ | QSLVHSNGNTY | 45 | KVS | 46 | SQSTHVPWT | 47 |
| IBV2042 | QSLVHSNRNTY | 48 | KVS | 49 | SQSTHVPFT | 50 |
| IBV2046 | QSISNY | 51 | YAS | 52 | QQSNSWPFT | 53 |
| IBV2047 | QSISNY | 51 | YAS | 52 | QQSYSWPFT | 54 |
| IBV2051 | QSIGTS | 55 | YAS | 52 | QQSNSWPFT | 53 |
| IBV2048² | EDIYNR | 56 | GAT | 57 | QQYWSTPLT | 58 |
| IBV2040 | EDIYNR | 56 | GAT | 57 | HQYWSTPLT | 59 |
| IBV2050 | EDIYNR | 56 | GAT | 57 | QQYWSSPLT | 60 |
| IBV2054 | ENVGTY | 61 | GAS | 62 | GQSYNYPYT | 63 |
| IBV2049³ | QGISNY | 64 | YTS | 65 | QQYSKLPWT | 66 |
| IBV2058 | SSVNY | 67 | YTS | 65 | QQFTSSTWT | 68 |
| IBV_L1 | QSISDY | 69 | YAS | 52 | QNGHSFPLT | 70 |
| IBV2056 | SSVSSSY | 71 | STS | 72 | QQYSGYPFT | 73 |
| IBV2101 | GNIHNY | 74 | NAK | 75 | QHFWSTPWT | 76 |

¹IBV2039 shares the same VL CDR1, CDR2, and CDR3 sequences with IBV2041, IBV2043, IBV2044, IBV2045, and IBV2057.
²IBV2048 shares the same VL CDR1, CDR2, and CDR3 sequences with IBV2053.
³IBV2049 shares the same VL CDR1, CDR2, and CDR3 sequences with IBV2052.

In some embodiments, the VL region comprises: CDR1 sequence selected from SEQ ID NOs: 45, 48, 51, 55, 56, 61, 64, 67, 69, 71, or 74; CDR2 sequence selected from SEQ ID NOs: 46, 49, 52, 57, 62, 65, 72, or 75; and CDR3 sequence selected from SEQ ID NOs: 47, 50, 53, 54, 58, 59, 60, 63, 66, 68, 70, 73, or 76.

In some instances, the VH region comprises CDR1 sequence selected from SEQ ID NOs: 1, 15, 36, and 39; CDR2 sequence selected from SEQ ID NOs: 2, 16, 37, and 40; and CDR3 sequence selected from SEQ ID NOs: 3, 4, 5, 17, 38, and 41; and the VL region comprises CDR1, CDR2, and CDR3 sequences of SEQ ID NOs: 45-47.

In some cases, the VH region comprises CDR1, CDR2, and CDR3 sequences of SEQ ID NOs: 6, 2, and 7; and the VL region comprise CDR1, CDR2, and CDR3 sequences of SEQ ID NOs: 48-50.

In some cases, the VH region comprises CDR1, CDR2, and CDR3 sequences of SEQ ID NOs: 23, 21, and 24; and the VL region comprise CDR1, CDR2, and CDR3 sequences of SEQ ID NOs: 51-53.

In some cases, the VH region comprises CDR1, CDR2, and CDR3 sequences of SEQ ID NOs: 18, 21, and 22; and the VL region comprise CDR1, CDR2, and CDR3 sequences of SEQ ID NOs: 51, 52, and 54.

In some cases, the VH region comprises CDR1, CDR2, and CDR3 sequences of SEQ ID NOs: 18-20; and the VL region comprise CDR1, CDR2, and CDR3 sequences of SEQ ID NOs: 55, 52, and 53.

In some cases, the VH region comprises CDR1 sequence selected from SEQ ID NO: 25, CDR2 sequence selected from SEQ ID NOs: 26 and 28, and CDR3 sequence selected from SEQ ID NO: 27, and the VL region comprise CDR1, CDR2, and CDR3 sequences of SEQ ID NOs: 56-58.

In some cases, the VH region comprises CDR1, CDR2, and CDR3 sequences of SEQ ID NOs: 25, 28, and 27; and the VL region comprise CDR1, CDR2, and CDR3 sequences of SEQ ID NOs: 56, 57, and 59.

In some cases, the VH region comprises CDR1, CDR2, and CDR3 sequences of SEQ ID NOs: 25, 29, and 27; and the VL region comprise CDR1, CDR2, and CDR3 sequences of SEQ ID NOs: 56, 57, and 60.

In some cases, the VH region comprises CDR1, CDR2, and CDR3 sequences of SEQ ID NOs: 30-32; and the VL region comprise CDR1, CDR2, and CDR3 sequences of SEQ ID NOs: 61-63.

In some cases, the VH region comprises CDR1 sequence selected from SEQ ID NO: 11, CDR2 sequence selected from SEQ ID NO: 12, and CDR3 sequence selected from SEQ ID NOs: 13 and 14, and the VL region comprise CDR1, CDR2, and CDR3 sequences of SEQ ID NOs: 64-66.

In some cases, the VH region comprises CDR1, CDR2, and CDR3 sequences of SEQ ID NOs: 8-10; and the VL region comprise CDR1, CDR2, and CDR3 sequences of SEQ ID NOs: 67, 65, and 68.

In some cases, the VH region comprises CDR1, CDR2, and CDR3 sequences of SEQ ID NOs: 42-44; and the VL region comprise CDR1, CDR2, and CDR3 sequences of SEQ ID NOs: 69, 52, and 70.

In some cases, the VH region comprises CDR1, CDR2, and CDR3 sequences of SEQ ID NOs: 33-35; and the VL region comprise CDR1, CDR2, and CDR3 sequences of SEQ ID NOs: 71-73.

In some cases, the VH region comprises CDR1, CDR2, and CDR3 sequences of SEQ ID NOs: 11-13; and the VL region comprise CDR1, CDR2, and CDR3 sequences of SEQ TD NOs: 74-76.

In some embodiments, an anti-CD47 antibody comprises a VH region and a VL region in which the sequence of the VH region comprises about 80%, 85%, 90%, 95%, 96%, 97%, 98%, 99%, or 100% sequence identity to SEQ ID NOs: 77, 79, 81, 83, 85, 87, 89, 91, 93, 95, 97, 99, 101, 103, 105, 107, 109, 111, 113, or 115; and the sequence of the VL region comprises about 80%, 85%, 90%, 95%, 96%, 97%, 98%, 99%, or 100% sequence identity to SEQ ID NOs: 78, 80, 82, 84, 86, 88, 90, 92, 94, 96, 98, 100, 102, 104, 106, 108, 110, 112, 114, or 116.

In some embodiments, the VH and VL region each independently comprises a sequence selected from Table 3. The underlined regions denote the respective CDR1, CDR2, or CDR3 sequence.

| | | SEQ | | SEQ ID |
|---|---|---|---|---|
| NAME | VH Sequence | ID NO: | VL Sequence | NO: |
| IBV2039 | EVQLQQSGAELVRSGASVKLS | 77 | DVVMTQTPLSLPVSLGDQ | 78 |
| | CTASGFNIKDYYIHWVKQRPE | | ASISCRSSQSLVHSNGNTY | |
| | QGLEWIGWIDPENGDTEYVPK | | LHWFLQKPGQSPKLLIYKV | |
| | FQGKATMTADTSSNTAYLQLS | | SNRFSGVPDRFSGSGSGTD | |
| | SLTSEDTAVYYCNAWGGSSSA | | FTLKISRVEAEDLGVYFCS | |
| | WFAYWGQGTLVTVSA | | QSTHVPWTFGGGTKLEIK | |
| IBV2040 | QVQLQQPGAELVRPGASVKLS | 79 | DIQMTQSSSSFSVSLGDRV | 80 |
| | CKASGYTFTSNWMNWVKQRP | | TITCKASEDIYNRLAWYQQ | |
| | EQGLEWIGRIDPYDSETHYNQK | | KPGNAPRLLISGATSFETG | |
| | FKDKAILTVDKSSNTAYMQLSS | | VPSRFSGSGSGKDYTLSITS | |
| | LTSEDSAVYYCARYYRYDGYF | | LQTEDVATYYCHQYWSTP | |
| | DYWGQGTTLTVSS | | LTFGAGTKLELK | |
| IBV2041 | EVQLQQSGAELVRSGASVKLS | 81 | DVVMTQTPLSLPVSLGDQ | 82 |
| | CTASGFNIKDYYMHWVKQRPE | | ASISCRSSQSLVHSNGNTY | |
| | QGLEWIGWIDPENGDTENAPK | | LHWNLQKPGQSPKLLIYK | |
| | FQGKATMTADTSSNTAYLHLS | | VSNRFSGVPDRFSGSGSGT | |
| | SLTSEDTAVYYCNAWGGNYPA | | DFTLKISRVEAEDLGVYFC | |
| | WFAYWGQGTLVTVSA | | SQSTHVPWTFGGGTKLEIK | |
| IBV2042 | EVQLQQSGAELVRSGASVKLS | 83 | DVVMTQSPLSLPVSLGDQ | 84 |
| | CTASGFNIKDHYMHWVKQRPE | | ASISCRSSQSLVHSNRNTY | |
| | QGLEWIGWIDPENGDTEYVPK | | LHWYLQKPGQSPKLLIYK | |
| | FQGKATMTADTSSNTAYLQLS | | VSNRFSGVPDRFSGSGSGT | |
| | SLTYEDTAVYYCNAGLFDYW | | DFTLKISRVEAEDLGVYFC | |
| | GQGTTLTVSS | | SQSTHVPFTFGSGTKLEIK | |
| IBV2043 | EVQLQQSGAELVRSGASVKLS | 85 | DVVMTQTPLSLPVSLGDQ | 86 |
| | CTASGFNIKDYYMHWVKQRPE | | ASISCRSSQSLVHSNGNTY | |
| | QGLEWIGWIDPENGDTECAPK | | LHWYLQKPGQSPKLLIYK | |
| | FQGKATMTADTSSNTAYLQLS | | VSNRFSGVPDRFSGSGSGT | |
| | SLTSEDTAVYYCNAWLERAM | | DFTLKISRVEAEDLGVYFC | |
| | DYWGQGTSVTVSS | | SQSTHVPWTFGGGTKLKIK | |
| IBV2044 | QVQLQQPGAELVMPGASVKM | 87 | DVVMTQTPLSLPVSLGDQ | 88 |
| | SCKASGYTFTDYWMHWVKQR | | ASISCRSSQSLVHSNGNTY | |
| | PGQGLEWIGAIDTSDSYTRYNQ | | LHWYLQKPGQSPKLLIYK | |
| | KFKGKATLTVDESSSTAYMQL | | VSNRFSGVPDRFSGSGSGT | |
| | SSLTSEDSAVYYCARGDYAMN | | DFTLKISRVEAEDLGVYFC | |
| | YWGQGTSVTVSS | | SQSTHVPWTFGGGTKLEIK | |
| IBV2045 | QVQLQQAGAELVMPGASVKM | 89 | DVVMTQTPLSLPVSLGDQ | 90 |
| | SCKASGHTFTDYWMHWVKQR | | ASISCRSSQSLVHSNGNTY | |
| | PGQGLEWIGAIDNSDSYTSYNQ | | LHWYLQKPGQSPKLLIYK | |
| | KFKDKATLTVDESSSTAHMLL | | VSNRFSGVPDRFSGSGSGT | |
| | SSLTSEDSAVYYCAKGYYAMD | | DFTLKISRVEAEDLGVYFC | |
| | YWGQGTSVTVSS | | SQSTHVPWTFGGGTKLEIK | |
| IBV2046 | EVQLQQSGPELVKPGASVKISC | 91 | DIVLTQSPATLSVTPGDRV | 92 |
| | KTSGYTFTEYIMHWVKQSHGK | | SLSCRASQSISNYLHWYQQ | |
| | SLEWIGGINPNNGGTSYNQKFK | | KSHESPRLLIKYASQSISGIP | |
| | GKATLTVDKSSSTAYMELRSL | | SRFNGSGSGTDFTLSINSVE | |
| | TSEDSAVYYCARRAYFDYWG | | TEDFGMFFCQQSNSWPFTF | |
| | QGTTLTVSS | | GSGTKLEIK | |
| IBV2047 | EVQLQQSGPELVKPGASVKISC | 93 | DIVLTQSPATLSVTPGDRV | 94 |
| | KTSGYTFTEYTMHWVKQSHG | | SLSCRASQSISNYLHWYQQ | |
| | KSLEWIGGINPNNGGTTYNQKF | | KSHESPRLLIKYASQSISGIP | |
| | KGKATLTVDKSSSTAYMEFRS | | SRFSGSGSGTDFTLSINSVE | |
| | LTSEDSAVYYCGRRSYFDYWG | | TEDFGMYFCQQSYSWPFT | |
| | QGTTLTVSS | | FGSGTKLEIK | |
| IBV2048 | QVQLQQPGAELVRPGASVKLS | 95 | DIQMTQSSSSFSVSLGDRV | 96 |
| | CKASGYTFTSNWMNWVKQRP | | TITCKASEDIYNRLAWYQQ | |
| | EQGLEWIGRIDPYDCETHYNQ | | KPGNAPRLLISGATSLETG | |
| | KFKDKAILTVDKSSNTAYMQL | | VPSRFSGSGSGKDYTLSITS | |
| | SSLTSEDSAVYYCARYYRYDG | | LQTEDVATYYCQQYWSTP | |
| | YFDYWGQGTTLTVSS | | LTFGAGTKLELK | |
| IBV2049 | EVQLQQSGPELVKTGASVKISC | 97 | DIQMTQTTSSLSASLGDRV | 98 |
| | KASGYSFTGYYMHWVKQSHG | | TISCSASQGISNYLNWYQQ | |
| | KSLEWIGYISCYNGATSYNQKF | | KPDGTVKLLIYYTSSLHSG | |
| | KGKATFTVDTSSSTAYMQFNS | | VPSRFSGSGSGTDYSLTISN | |
| | LTSEDSAVYYCARRDFYGMDY | | VEPEDIATYYCQQYSKLP | |
| | WGQGTSVTVSS | | WTFGGGTKLEIK | |
| IBV2050 | QVQLQQPGAELVRPGASVKLS | 99 | DIQMTQSSSSFSVSLGDRV | 100 |
| | CKASGYTFTSNWMNWVKQRP | | TITCKASEDIYNRLAWYQQ | |
| | EQGLEWIGRIDPYDTETHYSQK | | KPGNAPRLLISGATSLETG | |
| | FKAKAILTVDKSSNTAYMQLSS | | VPSRFSGSGSGKDYTLSITS | |
| | LTSEDSAVYYCARYYRYDGYF | | LQTEDVATYYCQQYWSSP | |
| | DYWGQGTTLTVSS | | LTFGAGTKLELK | |
| IBV2051 | EVQLQQSGPELVKPGASVKISC | 101 | DILLTQSPAILSVSPGEGVS | 102 |
| | KTSGYTFTEYTIHWVKQSHGK | | FSCRASQSIGTSIHWYQQR | |
| | SLEWIGGINPNIGATTYNQKFK | | TNGSPRLLIKYASESVSGIP | |
| | GKATLTVDKSSSTAYMELRSL | | SRFSGSGSGTGFTLSINSVE | |
| | TSEDSAVYYCARRAYFDYWG | | SEDIADYYCQQSNSWPFTF | |
| | QGTTLTVSS | | GSGTKLEIK | |
| IBV2052 | EVQLQQSGPELVKTGASVKISC | 103 | DIQMTQTTSSLSASLGDRV | 104 |
| | KASGYSFTGYYMHWVKQSHG | | TISCSASQGISNYLNWYQQ | |
| | KSLEWIGYISCYNGATSYNQKF | | KPDGTVKLLIYYTSSLHSG | |
| | KGKATFTVDTSSSTAYMQFNS | | VPSRFSGSGSGTDYSLTISN | |
| | LTSEDSAVYYCARRDFYAMDY | | LEPEDIATYYCQQYSKLPW | |
| | WGQGTSVTVSS | | TFGGGTKLEIK | |
| IBV2053 | QVQLQQPGAELVRPGASVKLS | 105 | DIQMTQSSSSFSVSLGDRV | 106 |
| | CKASGYTFTSNWMNWVKQRP | | TITCKASEDIYNRLAWYQQ | |
| | EQGLEWIGRIDPYDSETHYNQK | | KPGNAPRLLISGATSLETG | |
| | FKDKAILTVDKSSNTAYMQLSS | | VPSRFSGSGSGKDHTLSITS | |
| | LTSEDSAVYYCARYYRYDGYF | | LQTEDVATYYCQQYWSTP | |
| | DYWGQGTTLTVSS | | LTFGAGTKLELK | |
| IBV2054 | QVQLQQPGAELVRPGASVKLS | 107 | NIVMTQSPKSMSMSVGER | 108 |
| | CKASGYTFTSHWMNWVKQRP | | VTLSCKASENVGTYVSWF | |
| | GQGLEWIGMIDPSDSETHYNQ | | QQRPDQSPKLLIYGASNRY | |
| | MFKDKATLTVDKSSSTAYMQL | | TGVPDRFTGSGSATDFTLTI | |
| | SSLTSEDSAVYYCARLGRAWF | | SSVQAEDLADYHCGQSYN | |
| | AYWGQGTLVTVSA | | YPYTFGGGTKLEIK | |
| IBV2056 | QVQLQQSGAELMKPGASVKIS | 109 | ENVLTQSPAIMSASPGEKV | 110 |
| | CKATGYTFSSYWIQWVKQRPG | | TMTCRASSSVSSSYLHWY | |
| | HGLEWIGGILPGSGSTNYNEKF | | QQKSGASPKLWIYSTSNLA | |
| | KGKATFTADTSSNTAYMQLSS | | SGVPARFSGSGSGTSYSLTI | |
| | LTSEDSAVYYCARRGLLRFDV | | SSVEAEDAATYYCQQYSG | |
| | WGAGTTVTVSS | | YPFTFGSGTKLEIK | |
| IBV2057 | QVQLQQPGAEVVMPGASVKM | 111 | DVVMTQTPLSLPVSLGDQ | 112 |
| | SCKASGYTFTDYWMHWVKQR | | ASISCRSSQSLVHSNGNTY | |
| | PGQGLEWIGAIDTSDSYTSYNQ | | LHWYLQKPGQSPKLLIYK | |
| | KFKGKATLTVDESSSTAYMQL | | VSNRFSGVPDRFSGSGSGT | |
| | SSLTSEDSAVYYCARLGTGVA | | DFTLKISRVEAEDLGVYFC | |
| | TDYWGQGTTLTVSS | | SQSTHVPWTFGGGTKLEIK | |
| IBV2058 | DVQLQQSGAELVKPGASVKLS | 113 | ENVLTQSPAIMSASLGEKV | 114 |
| | CTASGFNIKDTYMHWVKQRPE | | TMSCRASSSVNYMYWYQ | |
| | QGLEWIGRIDPANGNTKYDPK | | QKSDASPKLWIYYTSNLAP | |
| | FQGKATITADTSSNTAYLQLSS | | GVPARFSGSGSGNSYSLTIS | |
| | LTSEDTAVYYCGRGDYYAMD | | SMEGEDAATYYCQQFTSS | |
| | YWGQGTSVTVSS | | TWTFGGGTKLEIK | |
| IBV2101 | EVQLQQSGPELVKTGASVKISC | 115 | DIQMTQSPASLSASVGETV | 116 |
| | KASGYSFTGYYIHWVKQSHGK | | TITCRASGNIHNYLAWYQ | |
| | SLEWIGYISCYNGATSYNQKFK | | QKQGKSPQLLVYNAKTLA | |
| | GKATFTVDTSSSTGYMQFNSLT | | DGVPSRFSGSGSGTQYSLK | |
| | SEDSAVYYCARRDFYGMDYW | | INSLQPEDFGSYYCQHFWS | |
| | GQGTSVTVSS | | TPWTFGGGTKLEIK | |

In some embodiments, an anti-CD47 antibody comprises a VH region and a VL region in which the sequence of the VH region comprises about 80%, 85%, 90%, 95%, 96% 97%, 98%, 99%, or 100% sequence identity to SEQ ID NOs: 117-119 and the sequence of the VL region comprises about 80%, 85%, 90%, 95%, 96% 97%, 98%, 99%, or 100% sequence identity to SEQ ID NOs: 120-122. In some cases, the anti-CD47 antibody comprises a VH region and a VL region in which the sequence of the VH region comprises about 80%, 85%, 90%, 95%, 96%, 97%, 98%, 99%, or 100% sequence identity to SEQ TD NO: 117. In some cases, the anti-CD47 antibody comprises a VH region and a VL region in which the sequence of the VH region comprises about 80%, 85%, 90%, 95%, 96% 97%, 98%, 99%, or 100% sequence identity to SEQ ID NO: 118. In some cases, the anti-CD47 antibody comprises a VH region and a VL region in which the sequence of the VH region comprises about 80%, 85%, 90%, 95%, 96%, 97%, 98%, 99% or 100% sequence identity to SEQ ID NO: 119. In some cases, the anti-CD47 antibody comprises a VH region and a VL region in which the sequence of the VL region comprises about 80%, 85%, 90%, 95%, 96%, 97%, 98%, 99%, or 100% sequence identity to SEQ ID NO: 120. In some cases, the anti-CD47 antibody comprises a VH region and a VL region in which the sequence of the VL region comprises about 80%, 85%, 90%, 95%, 96% 97%, 98%, 99%, or 100% sequence identity to SEQ ID NO: 121. In some cases, the anti-CD47 antibody comprises a VH region and a VL region in which the sequence of the VL region comprises about 80%, 85%, 90%, 95%, 96% 97%, 98%, 99%, or 100% sequence identity to SEQ ID NO: 122.

In some embodiments, the VH and VL region each independently comprises a sequence selected from Table 4. The underlined regions denote the respective CDR1, CDR2, or CDR3 sequence.

| NAME | SEQUENCE | SEQ ID NO: |
|---|---|---|
| IBV_VH1 | QVQLVQSGAEVKKPGASVKVSCKASGYAFTNYLIEWVRQAPGQGLE | 117 |
| | WMGVINPGSGATNYNQKFQGRVTMTADKSISTAYMELSRLRSDDTAV | |
| | YFCARRVYFDYWGQGTLVTVSS | |
| IBV_VH2 | QVQLVQSGAEVKKPGASVKVSCKASGYAFTNYLIEWVRQAPGQGLE | 118 |
| | WIGVINPGSGATNYNQKFQGRVTLTADKSISTAYMELSRLRSDDTAVY | |
| | FCARRVYFDYWGQGTLVTVSS | |
| IBV_VH3 | QVQLVQSGAEVKKPGASVKVSCKASGYAFTNYLIEWVRQAPGQGLE | 119 |
| | WMGVINPGSGATNYNQKFQGRVTMTADKSTSTVYMELSSLRSEDTAV | |
| | YFCARRVYFDYWGQGTLVTVSS | |
| IBV_VL1 | EIVLTQSPATLSLSPGERATLSCRASQSISDYLHWYQQKPGQAPRLLIK | 120 |
| | YASQSITGIPARFSGSGSGTDFTLTISSLEPEDFAVYYCQNGHSFPLTF | |
| | GQGTKLEIK | |
| IBV_VL2 | EIVMTQSPATLSLSPGERATLSCRASQSISDYLHWYQQKPGQAPRLLIK | 121 |
| | YASQSISGIPARFSGSGSGSDFTLTISSLEPEDFAVYYCQNGHSFPLTF | |
| | GQGTKLEIK | |
| IBV_VL3 | DIVMTQSPATLSLSPGERATLSCRASQSISDYLHWYQQKPGQAPRLLIK | 122 |
| | YASQSISGIPARFSGSGSGTDFTLTISSLEPEDFAVYYCQNGHSFPLTF | |
| | GQGTRLEIK | |

In some embodiments, an anti-CD47 antibody described herein is a full-length antibody. In other embodiments, the anti-CD47 antibody is a binding fragment. In some instances, the anti-CD47 antibody comprises a humanized antibody or binding fragment thereof, a chimeric antibody or binding fragment thereof, a monoclonal antibody or binding fragment thereof, or a bispecific antibody or binding fragment thereof. In some cases, the anti-CD47 antibody comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the anti-CD47 antibody comprises a bispecific antibody or binding fragment thereof. In some instances, the bispecific antibody or binding fragment thereof is a bispecific antibody conjugate, a hybrid bispecific IgG, a variable domain only bispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion.

In some embodiments, a bispecific antibody or binding fragment includes a Knobs-into-Holes (KiH), Asymmetric Re-egnineering Technology-immunoglobulin (ART-Ig), Triomab quadroma, bispecific monoclonal antibody (BiMAb, BsmAb, BsAb, bsMab, BS-Mab, or BiMAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody.

In some embodiments, a variable domain only bispecific antibody comprises a tandem scFv (taFv), triplebody, diabody (db), dsDb, db(KiH), scDb, dsFv-dsFv′, tandAbs, triple heads, tandem dAb/VHH, triple dAb/VHH, or tetravalent dAb/VHH.

In some embodiments, a CH1/CL fusion protein comprises a scFv2-CH1/CL or VHH2-CH1/CL.

In some instances, Fab fusion protein comprises a Fab-scFv (bibody), Fab-scFv2 (tribody), Fab-Fv, Fab-dsFv, Fab-VHH, or orthogonal Fab-Fab.

In some instances, a non-immunoglobulin fusion protein comprises a scFv2-albumin, scDb-albumin, taFv-albumin, taFv-toxin, miniantibody, DNL-Fab2, DNL-Fab2-scFv, DNL-Fab2-IgG-cytokine2, or ImmTAC (TCR-scFv).

In some instances, a Fc-modified IgG comprises a IgG (KiH), IgG (KiH) common LC, ZW1 IgG common LC, Biclonics common LC, CrossMAb, scFab-IgG (KiH), Fab-scFab-IgG (KiH), orthogonal Fab IgG (KiH), DuetMab, CH3 charge pair+CH1/CL charge pair, hinge/CH3 charge pair, DuoBody, four-in-one-CrossMab (KiH), LUZ-Y common LC, LUZ-Y scFab-IgG, or FcFc*.

In some instances, an appended & Fc-modified IgG comprises an IgG(KiH)-Fv, IgG(HA-TF-FV), IgG(KiH)-scFab, scFab-Fc(KiH)-scFv2, scFab-Fc(KiH)-scFv, half DVD-Ig, Dual Variable Domain-immunoglobulin (DVD-Ig), or CrossMab-Fab.

In some cases, a modified Fc and CH3 fusion protein comprises a scFv-Fc (KiH), scFv-Fc (CH3 charge pair), scFv-FC (EW-RVT), scFv-Fc (HA-TF), scFv-Fc (SEEDbody), taFv-Fc(KiH), scFv-Fc(KiH)-Fv, Fab-Fc(KiH)-scFv, Fab-scFv-Fc(KiH), Fab-scFv-Fc(BEAT), DART-Fc, scFv-CH3(KiH), or TriFabs.

In some cases, an appended IgG-HC fusion antibody comprises IgG-HC-scFv, IgG-dAb, IgG-taFv, IgG-CrossFab, IgG-orthogonal Fab, IgG-(CaCO) Fab, scFv-HC-IgG, tandem Fab-IgG, Fab-IgG(CaCpFab), Fab-IgG(CR3), or Fab-hinge-IgG(CR3).

In some cases, an appended IgG-LC fusion antibody comprises IgG-scFv(LC), scFv(LC)-IgG, or dAb-IgG.

In some cases, a non-immunoglobulin fusion antibody comprises DNL-Fab4-IgG.

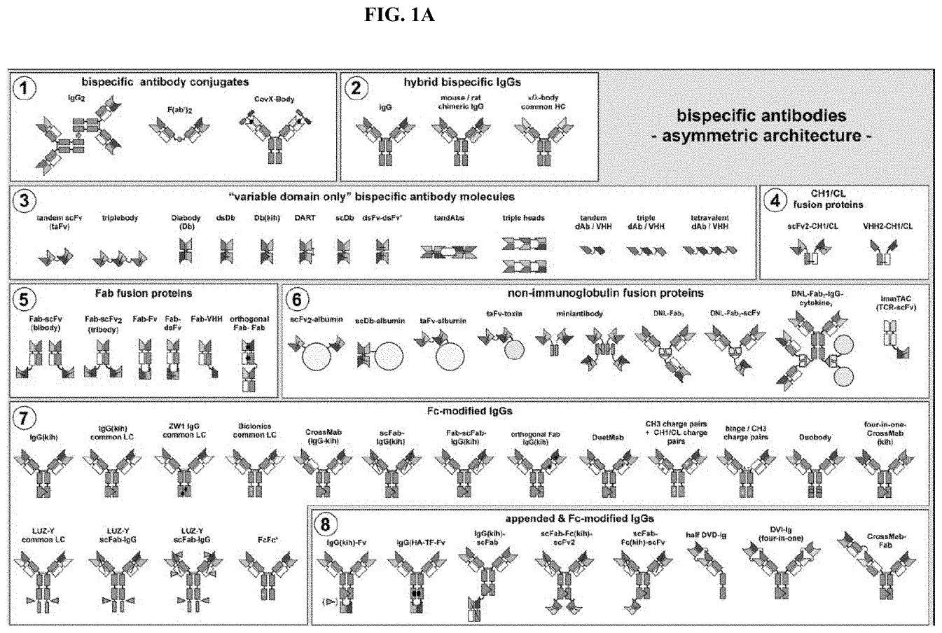

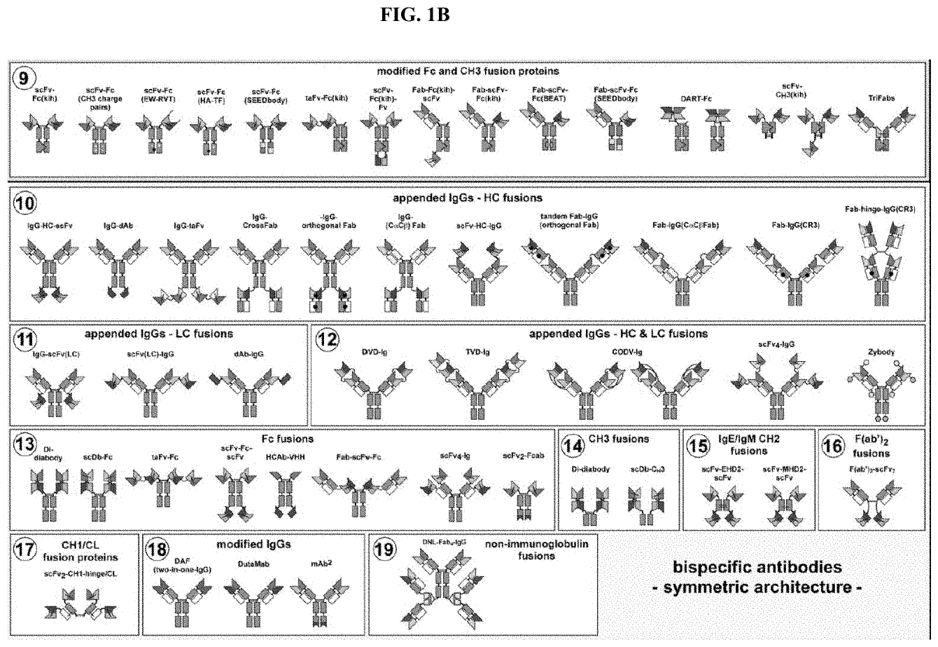

In some instances, the anti-CD47 antibody comprises a bispecific antibody or binding fragment thereof as illustrated in FIG. 1A or FIG. 1B.

In some embodiments, an anti-CD47 antibody described herein comprises a scFv that is further conjugated to an additional polypeptide comprising a hinge region, a transmembrane domain, a co-stimulatory domain (e.g., CD28, 4-1BB, CD27, or others) and a CD3 (activation domain to generate a chimeric antigen receptor (CAR) expressed on a T cell.

In some embodiments, an anti-CD47 antibody described herein comprises an IgG framework, an IgA framework, an IgE framework, or an IgM framework. In some instances, the anti-CD47 antibody comprises an IgG framework (e.g., IgG1, IgG2, IgG3, or IgG4). In such instances, the anti-CD47 antibody comprises an IgG1, IgG2, IgG3, or an IgG4 framework.

In some cases, the anti-CD47 antibody further comprises a mutation in a framework region. In some instances, the mutation is in the CH2 or CH3 region. In other instances, the mutation is in the hinge region. In some cases, the mutation is to stabilize the antibody and/or to increase half-life. In some cases, the mutation is to modulate Fc receptor interactions, e.g., to reduce or eliminate Fc effector functions such as FcγR, antibody-dependent cell-mediated cytotoxicity (ADCC), or complement-dependent cytotoxicity (CDC). In additional cases, the mutation is to modulate glycosylation.

In some cases, the anti-CD47 antibody comprises an IgG1 framework. In some embodiments, the constant region of the anti-CD47 antibody is modified at one or more amino acid positions to alter Fc receptor interaction. In some instances, the one or more amino acid positions comprise E233, L234, L235, G236, T250, M252, S254, T256, K322, A327, A330, P331, M428, H433, or N434 (Kabat numbering; EU index of Kabat et al 1991 Sequences of Proteins of Immunological Interest). In some instances, the mutation comprises E233P, L234A, L234V, L235E, L235A, G236A, T250Q, M252Y, S254T, T256E, K322A, A327G, A330S, P331S, M428L, H433K, or N434F.

In some embodiments, the modification at one or more amino acid positions in the IgG1 constant region to alter Fc receptor interaction leads to increased half-life. In some instances, the modification at one or more amino acid positions comprise T250, M252, S254, T256, M428, H433, N434, or a combination thereof, e.g., comprising T250Q/M428L or M252Y/S254T/T256E and H433K/N434F.

In some embodiments, the modification at one or more amino acid positions in the IgG1 constant region to alter Fc receptor interaction leads to reduced ADCC and/or CDC. In some instances, the modification at one or more amino acid positions comprise E233, L234, L235, G236, A327, K322, A330, P331, or a combination thereof. In some cases, the modification at one or more amino acid positions for reduced ADCC and CDC comprises, e.g., the combination E233P/L234V/L235A/G236 and A327G/A330S/P331S. In some cases, the modification at one or more amino acid positions for reduced CDC comprises K332A.

In some embodiments, the modification at one or more amino acid positions in the IgG1 constant region to alter Fc receptor interaction leads to increased macrophage phagocytosis. In some instances, the modification at one or more amino acid positions comprises G236, S239, 1332, or a combination thereof. In some cases, the modification at one or more amino acid positions for increased macrophage phagocytosis comprises the combination S239D/I332I/G236A.

In some embodiments, the IgG1 constant region is modified at amino acid N297 (Kabat numbering) to prevent glycosylation of the antibody. In some cases, the modification is N297A.

In some embodiments, the anti-CD47 antibody comprises an IgG2 framework. In some embodiments, the constant region of the anti-CD47 antibody is modified at one or more amino acid positions to alter Fc receptor interaction. In some instances, the one or more amino acid positions comprise L235, E318, K320, or K322 (Kabat numbering). In some cases, the mutation comprises L235E, E318A, K320A, or K322A.

In some instances, the modification at one or more amino acid positions in the IgG2 constant region to alter Fc receptor interaction leads to reduced ADCC and CDC. In some instances, the modification at one or more amino acid positions comprises L235, E318, K320, K322, or a combination thereof. In some cases, the modification at one or more amino acid positions for reduced ADCC and CDC comprises, e.g., the combination L235E and E318A/K320A/K322A.

In some cases, the IgG2 constant region is modified at amino acid N297 (Kabat numbering) to prevent to glycosylation of the antibody. In some cases, the modification is N297A

In some embodiments, the anti-CD47 antibody comprises an IgG3 framework. In some instances, the constant region of the antibody is modified at amino acid R435 to extend the half-life, e.g., R435H (Kabat numbering). In some cases, the constant region is further modified at amino acid N297 (e.g., N297A) to prevent to glycosylation of the antibody.

In some embodiments, the anti-CD47 antibody comprises an IgG4 framework. In some embodiments, the constant region of the antibody is modified at a hinge region to prevent or reduce strand exchange. In some instances, the amino acid that is modified is S228 (e.g., S228P). In some cases, the constant region is further modified at amino acid L235, e.g., S228P/L235E; at amino acid N297, e.g., S228P/N297A; or at amino acid positions S228, L235, and N297 (e.g., S228P/L235E/N297A).

In some embodiments, the human IgG constant region is modified to alter antibody-dependent cellular cytotoxicity (ADCC) and/or complement-dependent cytotoxicity (CDC), e.g., with a amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the human IgG constant region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some cases, heterodimerization via CH3 modifications is further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In some instances, an anti-CD47 antibody described herein lacks glycosylation, but is not modified at amino acid Asn297 (Kabat numbering). In these instances, the glycosylation is, for example, eliminated by production of the antibody in a host cell that lacks a post-translational glycosylation capacity, for example a bacterial or yeast derived system or a modified mammalian cell expression system. In certain aspects, such a system is a cell-free expression system.

In some embodiments, the heavy chain (HC) comprises a sequence selected from Table 5. In some embodiments, the light chain (LC) comprises a sequence selected from Table 6. The underlined region denotes the respective CDR1, CDR2, or CDR3 sequence.

TABLE 5

| | | SEQ ID |
|---|---|---|
| NAME | SEQUENCE | NO: |
| HC1 | MDPKGSLSWRILLFLSLAFELSYGQVQLVQSGAEVKKPGASVKVSCKASGYAF | 123 |
| | TNYLIEWVRQAPGQGLEWMGVINPGSGATNYNQKFQGRVTMTADKSISTAY | |
| | MELSRLRSDDTAVYFCARRVYFDYWGQGTLVTVSSASTKGPSVFPLAPCSRST | |
| | SESTAALGCLVKDYFPEPVTV SWNSGALTSGVHTFPAVLQSSGLYSLSSVVTV | |
| | PSSSLGTKTYTCNVDHKPSNTKVDKRVESKYGPPCPPCPAPEFLGGPSVFLFPP | |
| | KPKDTLMISRTPEVTCVVVDVSQEDPEVQFNWYVDGVEVHNAKTKPREEQFN | |
| | STYRVVSVLTVLHQDWLNGKEYKCKVSNKGLPSSIEKTISKAKGQPREPQVYT | |
| | LPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLG | |
| HC2 | MDPKGSLSWRILLFLSLAFELSYGQVQLVQSGAEVKKPGASVKVSCKASGYAF | 124 |
| | TNYLIEWVRQAPGQGLEWIGVINPGSGATNYNQKFQGRVTLTADKSISTAYME | |
| | LSRLRSDDTAVYFCARRVYFDYWGQGTLVTVSSASTKGPSVFPLAPCSRSTSE | |
| | STAALGCLVKDYFPEPVTVSWNSGALTSGVHTFPAVLQSSGLYSLSSVVTVPSS | |
| | SLGTKTYTCNVDHKPSNTKVDKRVESKYGPPCPPCPAPEFLGGPSVFLFPPKPK | |
| | DTLMISRTPEVTCVVVDVSQEDPEVQFNWYVDGVEVHNAKTKPREEQFNSTY | |
| | RVVSVLTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDSDGSFFL | |
| | YSRLTVDKSRWQEGNVFSCSVMHEALHNHYTQKSLSLSLG | |
| HC3 | MDPKGSLSWRILLFLSLAFELSYGQVQLVQSGAEVKKPGASVKVSCKASGYAF | 125 |
| | TNYLIEWVRQAPGQGLEWMGVINPGSGATNYNQKFQGRVTMTADKSTSTVY | |
| | MELSSLRSEDTAVYFCARRVYFDYWGQGTLVTVSSASTKGPSVFPLAPCSRST | |
| | SESTAALGCLVKDYFPEPVTVSWNSGALTSGVHTFPAVLQSSGLYSLSSVVTV | |
| | PSSSLGTKTYTCNVDHKPSNTKVDKRVESKYGPPCPPCPAPEFLGGPSVFLFPP | |
| | KPKDTLMISRTPEVTCVVVDVSQEDPEVQFNWYVDGVEVHNAKTKPREEQFN | |
| | STYRVVSVLTVLHQDWLNGKEYKCKVSNKGLPSSIEKTISKAKGQPREPQVYT | |
| | LPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLG | |

TABLE 6

| | | SEQ ID |
|---|---|---|
| Name | SEQUENCE | NO: |
| LC1 | METDTLLLWVLLLWVPGSTGEIVLTQSPATLSLSPGERATLSCRASQSISDYLH | 126 |
| | WYQQKPGQAPRLLIKYASQSITGIPARFSGSGSGTDFTLTISSLEPEDFAVYYCQ | |
| | NGHSFPLTFGQGTKLEIKRTVAAPSVFIFPPSDEQLKSGTASVVCLLNNFYPREA | |
| | KVQWKVDNALQSGNSQESVTEQDSKDSTYSLSSTLTLSKADYEKHKVYACEV | |
| | THQGLSSPVTKSFNRGEC | |
| LC2 | METDTLLLWVLLLWVPGSTGEIVMTQSPATLSLSPGERATLSCRASQSISDYLH | 127 |
| | WYQQKPGQAPRLLIKYASQSISGIPARFSGSGSGSDFTLTISSLEPEDFAVYYCQN | |
| | GHSFPLTFGQGTKLEIKRTVAAPSVFIFPPSDEQLKSGTASVVCLLNNFYPREAK | |
| | VQWKVDNALQSGNSQESVTEQDSKDSTYSLSSTLTLSKADYEKHKVYACEVT | |
| | HQGLSSPVTKSFNRGEC | |
| LC3 | METDTLLLWVLLLWVPGSTGDIVMTQSPATLSLSPGERATLSCRASQSISDYLH | 128 |
| | WYQQKPGQAPRLLIKYASQSISGIPARFSGSGSGTDFTLTISSLEPEDFAVYYCQ | |
| | NGHSFPLTFGQGTRLEIKRTVAAPSVFIFPPSDEQLKSGTASVVCLLNNFYPREA | |
| | KVQWKVDNALQSGNSQESVTEQDSKDSTYSLSSTLTLSKADYEKHKVYACEV | |
| | THQGLSSPVTKSFNRGEC | |

In some instances, an anti-CD47 antibody described herein promotes (e.g., induces or increases) phagocytosis of cells, e.g., CD47-expressing cells, for example, by macrophages. In such cases, the level of phagocytosis of a CD47-expressing cell in the presence of an anti-CD47 antibody described herein is increased by at least 5%, at least 10%, at least 20%, at least 30%, at least 40%, at least 50%, at least 60%, at least 70%, at least 80%, at least 90%, at least 99%, at least 150%, at least 200%, at least 500%, or more. In some instances, the level of phagocytosis is compared to the level of a phagocytosis of an equivalent CD47-expression cell in the presence of a reference antibody (e.g., CC-90002 or hu5F9).

In some cases, an anti-CD47 antibody described herein does not promote (e.g., induces or increases), or causes a significant level of, agglutination of cells, e.g., an anti-CD47 antibody described herein does not promote (e.g., induces or increases), or causes a significant level of, hemagglutination of red blood cells (RBCs). In some cases, the level of agglutination of a cell (e.g., a red blood cell) in the presence of an anti-CD47 antibody described herein is reduced by at least 5%, at least 10%, at least 20%, at least 30%, at least 40%, at least 50%, at least 60%, at least 70%, at least 80%, at least 90%, or at least 99% compared to the level of agglutination of an equivalent cell (e.g., an equivalent red blood cell) in the presence of a reference antibody (e.g., CC-90002). In some cases, an anti-CD47 antibody described herein does not promote (e.g., induces or increases), or causes a significant level of, agglutination if the level of agglutination in the presence of the anti-CD47 antibody is reduced by at least 5%, at least 10%, at least 20%, at least 30%, at least 40%, at least 50%, at least 60%, at least 70%, at least 80%, at least 90%, or at least 99% compared to the level of agglutination in the presence of a reference antibody (e.g., CC-90002).

In some aspects, the level of RBC depletion is determined by measuring the RBC count in a subject after administration of a treatment, e.g., an anti-CD47 antibody described herein. In some embodiments, anti-CD47 antibodies described herein do not promote (e.g., induce or increase), or cause a significant level of, RBC depletion if the RBC count in a subject after administration of an anti-CD47 antibody described herein is within the range of a normal, healthy subject. For example, the RBC count for a normal, healthy male human is about 4.7 to about 6.1 million cells per microliter of blood sample. For example, the RBC count for a normal, healthy female human is 4.2 to about 5.4 million cells per microliter of blood sample. In some aspects, anti-CD47 antibodies described herein do not promote (e.g., induce or increase), or cause a significant level of, RBC depletion if the RBC count in a subject after administration (e.g., 5 min, 10 min, 30 min, 1 h, 2 h, 3 h, 4 h, 5 h, 12 h, 24 h, 2 days, 4 days, 6 days, 1 week, 2 weeks, 3 weeks, 1 month, 2 months, or more) of an anti-CD47 antibody described herein is at least 50%, 60%, 70%, 80%, 90%, 95%, 97%, 98%, 99%, or 99.5% of the RBC count prior to administration. In some aspects, anti-CD47 antibodies described herein do not promote (e.g., induce or increase), or cause a significant level of, RBC depletion if the RBC count in a subject after administration (5 min, 10 min, 30 min, 1 h, 2 h, 3 h, 4 h, 5 h, 12 h, 24 h, 2 days, 4 days, 6 days, 1 week, 2 weeks, 3 weeks, 1 month, 2 months, or more) of an anti47 antibody described herein is at least 50%, 60%, 70%, 80%, 90%, 95%, 97%, 98%, 99%, or 99.5% of the RBC count in a subject after administration of a placebo treatment (e.g., vehicle). RBC counts are determined by standard methods in the art.

In some cases, an anti-CD47 antibody described herein does not promote (e.g., induces or increases), or causes a significant level of, platelet depletion. For example, administration of an anti-CD47 antibody described herein leads to a percentage of platelets remaining of at least 30%, 40%, 50%, 60%, 70%, 80%, 90%, or 100%.

Those skilled in the art will recognize that it is possible to quantitate, without undue experimentation, the level of agglutination, e.g., the level of hemagglutination of RBCs. For example, those skilled in the art will recognize that the level of hemagglutination is ascertained by measuring the area of an RBC dot after performing a hemagglutination assay in the presence of anti-CD47 antibodies described herein. In some cases, the area of the RBC dot in the presence of anti-CD47 antibody described herein is compared to the area of the RBC dot in the absence of an anti-CD47 antibody, e.g., in the presence of zero hemagglutination. In this manner, hemagglutination is quantified relative to a baseline control. A larger RBC dot area corresponds to a higher level of hemagglutination. Alternatively, densitometry of the RBC dot is optionally utilized to quantitate hemagglutination.

Those skilled in the art will recognize that it is possible to quantitate, without undue experimentation, the level of RBC depletion. For example, those skilled in the art will recognize that the level of RBC depletion is ascertained, e.g., by measuring the RBC count (i.e., the total number of RBCs in a sample of blood), e.g., by using a cell counter or a hemacytometer. Those of skill in the art will recognize that the RBCs in a sample of blood can optionally be isolated by fractionating whole blood using, e.g., centrifugation, prior to counting. In some cases, the RBC count in the presence of an anti-CD47 antibody described herein is compared to the RBC count in the absence of the CD47 antibody, e.g., in the presence of zero RBC depletion. In this manner, the level of RBC depletion is normalized relative to a baseline control.

In some embodiments, anti-CD47 antibodies provided herein exhibit inhibitory activity, for example by inhibiting CD47 expression (e.g., inhibiting cell surface expression of CD47), activity, and/or signaling, or by interfering with the interaction between CD47 and SIRPα. In some instances, anti-CD47 antibodies provided herein completely or partially reduce or otherwise modulate CD47 expression or activity upon binding to, or otherwise interacting with, CD47, e.g., a human CD47. The reduction or modulation of a biological function of CD47 is complete, significant, or partial upon interaction between the antibodies and the human CD47 polypeptide and/or peptide. Anti-CD47 antibodies described herein are considered to completely inhibit CD47 expression or activity when the level of CD47 expression or activity in the presence of the antibody is decreased by at least 95%, e.g., by 96%, 97%, 98%, 99%, 100% as compared to the level of CD47 expression or activity in the absence of interaction, e.g., binding, with the antibody described herein. In some cases, anti-CD47 antibodies are considered to significantly inhibit CD47 expression or activity when the level of CD47 expression or activity in the presence of the CD47 antibody is decreased by at least 50%, e.g., 55%, 60%, 75%, 80%, 85%, 90% as compared to the level of CD47 expression or activity in the absence of binding with a CD47 antibody described herein. In some cases, anti-CD47 antibodies are considered to partially inhibit CD47 expression or activity when the level of CD47 expression or activity in the presence of the antibody is decreased by less than 95%, e.g., 10%, 20%, 25%, 30%, 40%, 50%, 60%, 75%, 80%, 85%, 90% as compared to the level of CD47 expression or activity in the absence of interaction, e.g., binding, with an antibody described herein.

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad Sci. USA 77:357; O'Hare et al., 1981, Proc. Natl. Acad Sci. USA 78:1527); gpt, which confers resistance to mycophenolic acid (Mulligan & Berg, 1981, Proc. Natl. Acad Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi Bacteroidetes (FCB group), Fusobacteria, Gemmatimonadetes, Nitrospirae, Planctomycetes-Verrucomicrobia Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™ DH10B™, TOP10, DH5a, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS 115, KM71H, SMD 1168, SMD 1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cell include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

Payloads

In some embodiments, an anti-CD47 antibody described herein is further conjugated to one or more payloads. In some instances, the payload is a small molecule. In other instances, the payload is a protein or peptide. In additional instances, the payload is a nucleic acid polymer. In some cases, the payload is conjugated to the anti-CD47 antibody directly. In other cases, the payload is conjugated to the anti-CD47 antibody indirectly via a linker.

In some instances, the number of payloads conjugated to the anti-CD47 antibody (e.g., the drug-to-antibody ratio or DAR) is about 1:1, one payload to one anti-CD47 antibody. In some cases, the ratio of the payload to the anti-CD47 antibody is about 2:1, 3:1, 4:1, 5:1, 6:1, 7:1, 8:1, 9:1, 10:1, 11:1, or 12:1. In some cases, the ratio of the payload to the anti-CD47 antibody is about 2:1. In some cases, the ratio of the payload to the anti-CD47 antibody is about 4:1. In some cases, the ratio of the payload to the anti-CD47 antibody is about 8:1. In some cases, the ratio of the payload to the anti-CD47 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the tubulysin is a tubulysin analog or derivative such as described in U.S. Pat. Nos. 8,580,820 and 8,980,833 and in U.S. Publication Nos. 20130217638, 20130224228, and 201400363454.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714 and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload is an imaging agent. In some instances, the payload comprises a “radio-opaque” label, e.g. a label visualized using x-rays. Radio-opaque materials are well known to those of skill in the art. Exemplary radio-opaque materials include iodide, bromide or barium salts. Additional radiopaque materials include, but are not limited to, organic bismuth derivatives {see, e.g., U.S. Pat. No. 5,939,045), radio-opaque polyurethanes (see, e.g., U.S. Pat. No. 5,346,981), organobismuth composites (see, e.g., U.S. Pat. No. 5,256,334), radio-opaque barium polymer complexes (see, e.g., U.S. Pat. No. 4,866,132), and the like.

In some instances, the payload comprises a detectable label, for example, for use in immunoconjugates include any composition detectable by spectroscopic, photochemical, biochemical, immunochemical, electrical, optical or chemical means. Useful labels in the include magnetic beads (e.g., DYNABEADS™), fluorescent dyes (e.g., fluorescein isothiocyanate, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g. polystyrene, polypropylene, latex, etc.) beads, nanoparticles, quantum dots, and the like.

In some embodiments, suitable radiolabels include, but are not limited to, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag.

In some instances, the payload comprises a radiosensitizer that enhances the cytotoxic effect of ionizing radiation (e.g., such as might be produced by ⁶⁰Co or an x-ray source) on a cell. Numerous radiosensitizing agents are known and include, but are not limited to benzoporphyrin derivative compounds (see, e.g., U.S. Pat. No. 5,945,439), 1,2,4-benzotriazine oxides (see, e.g., U.S. Pat. No. 5,849,738), compounds containing certain diamines (see, e.g., U.S. Pat. No. 5,700,825), BCNT (see, e.g., U.S. Pat. No. 5,872,107), radiosensitizing nitrobenzoic acid amide derivatives (see, e.g., U.S. Pat. No. 4,474,814), various heterocyclic derivatives (see, e.g., U.S. Pat. No. 5,064,849), platinum complexes (see, e.g., U.S. Pat. No. 4,921,963), and the like.

In some instances, the payload comprises an alpha emitter, i.e. a radioactive isotope that emits alpha particles. Alpha-emitters have recently been shown to be effective in the treatment of cancer (see, e.g., McDevitt et al. (2001) Science 294: 1537-1540; Ballangrud et al. (2001) Cancer Res. 61: 2008-2014; Borchardt et al. (2003) Cancer Res. 63: 5084-50). Suitable alpha emitters include, but are not limited to ²¹³Bi, ²¹¹At, and the like.

In some embodiments, the payload comprises a protein or peptide toxin or fragment thereof. Exemplary enzymatically active toxins and fragments thereof include, but are not limited to, diphtheria toxin A fragment, nonbinding active fragments of diphtheria toxin, exotoxin A (from Pseudomonas aeruginosa), ricin A chain, abrin A chain, modeccin A chain, a-sacrin, certain A leurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, enomycin, and tricothecenes.

In some instances, the payload is an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some instances, the payload comprises a cytokine. In some embodiments, the cytokine comprises IL-2, IL-2, IL-3, IL-6, IL-10, IL-12, IL-18, IL-21, interferon (e.g., IFNα, IFNβ), or TNFα.

In some instances, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload is a nucleic acid polymer. In some instances, the nucleic acid polymers include those for use in gene therapy, such as in RNA interference (RNAi) or gene silencing (or antisense oligonucleotide) techniques. Exemplary nucleic acid polymers that participate in the RNA interference process include short interfering nucleic acid (siNA), short interfering RNA (siRNA), double-stranded RNA (dsRNA), micro-RNA (miRNA), or short hairpin RNA (shRNA).

Antisense oligonucleotides include nucleotide sequence that is substantially complementary to a target nucleotide sequence in e.g., a pre-mRNA molecule, hrRNA (heterogenous nuclear RNA), or mRNA molecule. The degree of complementarity (or substantial complementarity) of the antisense sequence is preferably such that a molecule comprising the antisense sequence form a stable hybrid with the target nucleotide sequence in the RNA molecule under physiological condition.

In some embodiments, the nucleic acid polymer is an mRNA. In some cases, the mRNA encodes a cytotoxic protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL-panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT).

In some instances, the mRNA encodes a cytotoxic peptide or peptide related to the immune system such as a cytotoxic T cell or B cell epitope to stimulate a specific immune response via presentation of such epitope with an MIIC I complex, an membrane attack complex protein (MAC) of the complement system, perforin, a granzyme and a granulysin.

In some cases, the mRNA encodes an apoptotic triggering protein or peptide such as an apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2).

In some embodiments, the nucleic acid polymer is a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some instances, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′—OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modification include morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof.

### Conjugation Chemistry

In some instances, the payload is conjugated to an anti-CD47 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-CD47 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-CD47 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-CD47 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-CD47 antibody utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-CD47 antibody described herein by a method as described in PCT Publication No. WO2014/140317, which utilizes a sequence-specific transpeptidase.

In some instances, the payload is conjugated to an anti-CD47 antibody described herein by a method as described in U.S. Patent Publication Nos. 2015/0105539 and 2015/0105540.

### Linkers

In some embodiments, the payload is conjugated to an anti-CD47 antibody described herein indirectly via a linker. In some instances, the linker comprises a natural or synthetic polymer, consisting of long chains of branched or unbranched monomers, and/or cross-linked network of monomers in two or three dimensions. In some instances, the linker includes a polysaccharide, lignin, rubber, or polyalkylen oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some cases, the linker is a dPEG from Quanta Biodesign, LMD.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(7-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some embodiments, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Methods of Use

In some embodiments, described herein are methods of inducing phagocytosis, methods of treating a disease or condition, and methods of dampening or decreasing an inflammatory response with use of an anti-CD47 antibody. In some instances, also described herein are methods of inhibiting, blocking, or disruption an interaction between CD47 and SIRPPα or CD47 and TSP-1 with the use of an anti-CD47 antibody.

In some instances, described herein is a method of inducing phagocytosis of a target cell expressing CD47 which comprises contacting the target cell with an anti-CD47 antibody described supra for a time sufficient for binding of the antibody to CD47, in which the anti-CD47 antibody blocks interaction of CD47 with SIRPPα expressed on a phagocytic cell (e.g., macrophage or dendritic cell), thereby inducing phagocytosis of the target cell. In some cases, the target cell is a tumor cell or a cell infected with a pathogen. In some cases, the target cell is a cancer cell, optionally from a solid tumor such as bladder cancer, bone cancer, brain cancer, breast cancer, colorectal cancer, eye cancer, head and neck cancer, kidney cancer, liver cancer, lung cancer, pancreatic cancer, prostate cancer, skin cancer (e.g., melanoma), stomach cancer, thyroid cancer, or uterine cancer; or from a hematologic malignancy such as chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the target cell is a pathogen-infected cell, such as an infected cell by a virus, a bacterium, or a protozoan.

In some instances, described herein is a method of decreasing or dampening an inflammatory response which comprises contacting a target cell with an anti-CD47 antibody described supra for a time sufficient for binding of the antibody to CD47, in which the anti-CD47 antibody blocks interaction of CD47 with TSP-1 expressed on a platelet, an endothelial cell, a smooth-muscle cell, a leukocyte, or an activated astrocyte to decrease or dampen the inflammatory response.

In some instances, also described herein is a method of treating a disease or condition in a subject in need thereof. In some instances, the disease or condition is a proliferative disease, an inflammatory disease, an ischemia-reperfusion injury, or a pathogenic infection. In some cases, the disease or condition is associated with the CD47-SIRPα signaling pathway. In additional cases, the disease or condition is associated with the CD47-TSP-1 signaling pathway.

In additional instances, further described herein is a use of an anti-CD47 antibody described above for treating a non-human animal disease. In such cases, the disease is associated with an overexpression of CD47 or a paralog thereof or an impaired function of CD47 or a paralog thereof. In additional cases, the non-human animal refers to any animals expressing CD47 or a paralog thereof, including but not limited to, dogs, cats, farm animals, and the like.

### Proliferative Diseases

In some embodiments, the disease or condition is a proliferative disease (e.g., cancer). In some instances, the method comprises administering to the subject an anti-CD47 antibody described supra, thereby treating the proliferative disease (e.g., cancer) in the subject. In some cases, the disease or condition is a cancer (e.g., a solid tumor or a hematologic malignancy).

In some embodiments, the cancer is a solid tumor. In some cases, the solid tumor is a tumor of the bones, muscle, or organs. In some cases, the solid tumor is a sarcoma or a carcinoma. Exemplary sarcomas include Ewing sarcoma, osteosarcoma, and rhabdomyosarcoma. Exemplary carcinoma include adenocarcinoma, basal cell carcinoma, squamous cell carcinoma, renal cell carcinoma, Ductal carcinoma in situ (DCIS), and invasive ductal carcinoma. In some cases, the solid tumor is bladder cancer, bone cancer, brain cancer, breast cancer, colorectal cancer, eye cancer, head and neck cancer, kidney cancer, liver cancer, lung cancer, ovarian cancer, pancreatic cancer, prostate cancer, skin cancer, stomach cancer, thyroid cancer, or uterine cancer. In some instances, the solid tumor is a metastatic solid tumor. In some instances, the solid tumor is a relapsed or refractory solid tumor.

In some embodiments, the cancer is a hematologic malignancy. In some cases, the hematologic malignancy is a B cell lymphoma or a T cell lymphoma. In some cases, the hematologic malignancy is a Hodgkin's lymphoma or a non-Hodgkin's lymphoma. In some cases, the hematologic malignancy is chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a metastatic hematologic malignancy. In additional cases, the hematologic malignancy is a relapsed or refractory hematologic malignancy.

In some embodiments, an anti-CD47 antibody described herein is administered in combination with an additional therapeutic agent for the treatment of cancer. In some instances, the additional therapeutic agent comprises chemotherapeutic agent, immunotherapeutic agent, targeted therapeutic agent, hormone-based therapeutic agent, stem-cell based therapeutic agent, or radiation. In some cases, the additional therapeutic agent is a first-line treatment. In some cases, exemplary therapeutic agents include, but are not limited to, anti-cancer antibodies (e.g., HERCEPTIN®), antimetabolites, alkylating agents, topoisomerase inhibitors, microtubule targeting agents, kinase inhibitors, protein synthesis inhibitors, somatostatin analogs, glucocorticoids, aromatose inhibitors, mTOR inhibitors, protein Kinase B (PKB) inhibitors, phosphatidylinositol, 3-Kinase (PI3K) Inhibitors, cyclin dependent kinase inhibitors, anti-TRAIL molecules, MEK inhibitors, and the like.

In some embodiments, the additional therapeutic agents include, but are not limited to flourouracil (5-FU), capecitabine/XELODA, 5-Trifluoromethyl-2′-deoxyuridine, methotrexate sodium, raltitrexed/Tomudex, pemetrexed/Alimta®, cytosine Arabinoside (Cytarabine, Ara-C)/Thioguanine, 6-mercaptopurine (Mercaptopurine, 6-MP), azathioprine/Azasan, 6-thioguanine (6-TG)/Purinethol (TEVA), pentostatin/Nipent, fludarabine phosphate/Fludara®, cladribine (2-CdA, 2-chlorodeoxyadenosine)/Leustatin, floxuridine (5-fluoro-2)/FUDR (Hospira, Inc.), ribonucleotide Reductase Inhibitor (RNR), cyclophosphamide/Cytoxan (BMS), neosar, ifosfamide/Mitoxana, thiotepa, BCNU-1,3-bis(2-chloroethyl)-1-nitosourea, 1,-(2-chloroethyl)-3-cyclohexyl-lnitrosourea, methyl CCNU, hexamethylmelamine, busulfan/Myleran, procarbazine HCL/Matulane, dacarbazine (DTIC), chlorambucil/Leukaran®, melphalan/Alkeran, cisplatin (Cisplatinum, CDDP)/Platinol, carboplatin/Paraplatin, oxaliplatin/Eloxitan, bendamustine, carmustine, chloromethine, dacarbazine (DTIC), fotemustine, lomustine, mannosulfan, nedaplatin, nimustine, prednimustine, ranimustine, satraplatin, semustine, streptozocin, temozolomide, treosulfan, triaziquone, triethylene melamine, thioTEPA, triplatin tetranitrate, trofosfamide, uramustine, doxorubicin HCL/Doxil, daunorubicin citrate/Daunoxome®, mitoxantrone HCL/Novantrone, actinomycin D, etoposide/Vepesid, topotecan HCL/Hycamtin, teniposide (VM-26), irinotecan HCL (CPT-11), Camptosar®, camptothecin, Belotecan, rubitecan, vincristine, vinblastine sulfate, vinorelbine tartrate, vindesine sulphate, paclitaxel/Taxol, docetaxel/Taxotere, nanoparticle paclitaxel, abraxane, ixabepilone, larotaxel, ortataxel, tesetaxel, vinfiunine, and the like.

In some embodiments the additional therapeutic agents comprise one or more drugs selected from the group consisting of carboplatin (e.g., PARAPLATIN®), Cisplatin (e.g., PLATINOL®, PLATINOL-AQ®), Cyclophosphamide (e.g., CYTOXAN®, NEOSAPv®), Docetaxel (e.g., TAXOTERE®), Doxorubicin (e.g., ADRIAMYCIN®), Erlotinib (e.g., TARCEVA®), Etoposide (e.g., VEPESID®), Fluorouracil (e.g., 5-FU®), Gemcitabine (e.g., GEMZAR®), imatinib mesylate (e.g., GLEEVEC®), Irinotecan (e.g., CAMPTOSAR®), Methotrexate (e.g., FOLEX®, MEXATE®, AMETHOPTERIN®), Paclitaxel (e.g., TAXOL®, ABRAXANE®), Sorafmib (e.g., NEXAVAR®), Sunitinib (e.g., SUTENT®), Topotecan (e.g., HYCAMTIN®), Vinblastine (e.g., VELBAN®), Vincristine (e.g., ONCOVIN®, VINCASAR PFS®). In some embodiments, the additional therapeutic agents comprises one or more drugs selected from the group consisting of retinoic acid, a retinoic acid derivative, doxorubicin, vinblastine, vincristine, cyclophosphamide, ifosfamide, cisplatin, 5-fluorouracil, a camptothecin derivative, interferon, tamoxifen, and taxol. In some instances, the additional therapeutic agent is selected from the group consisting of abraxane, doxorubicin, pamidronate disodium, anastrozole, exemestane, cyclophosphamide, epirubicin, toremifene, letrozole, trastuzumab, megestroltamoxifen, paclitaxel, docetaxel, capecitabine, goserelin acetate, zoledronic acid, vinblastine, etc.), an antisense molecule, an siRNA, and the like.

In some embodiments, the additional therapeutic agent comprises an immune checkpoint modulator. Exemplary checkpoint modulators include:

PD-L1 modulators such as Genentech's MPDL3280A (RG7446), Avelumab (Bavencio) from Merck/Pfizer, durvalumab (Imfinzi) from AstraZeneca, Anti-mouse PD-L1 antibody Clone 10F.9G2 (Cat #BE0101) from BioXcell, anti-PD-L1 monoclonal antibody MDX-1105 (BMS-936559), BMS-935559 and BMS-986192 from Bristol-Meyer's Squibb, MSB0010718C, mouse anti-PD-L1 Clone 29E.2A3, CX-072 from XytomX Therapeutics, FAZ053 from Novartis Pharmaceuticals, KN035 from 3D Medicine, LY3300054 from Eli Lilly, and AstraZeneca's MEDI4736;

PD-L2 modulators such as GlaxoSmithKline's AMP-224 (Amplimmune), and rHIgM12B7;

PD-1 modulators such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, sintilimab (IBI-308) from Eli Lilly/Innovent Biologics, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDIO680 from MedImmune, PDR001 from Novartis Pharmaceuticals, PF-06801591 from Pfizer, REGN2810 from Regeneron Pharmaceuticals, and Pidilizumab (CT-O11) from CureTech Ltd;

CTLA-4 modulators such as Bristol Meyers Squibb's anti-CTLA-4 antibody ipilimumab (also known as Yervoy®, MDX-010, BMS-734016 and MDX-101), anti-CTLA4 antibody clone 9H10 from Millipore, Pfizer's tremelimumab (CP-675,206, ticilimumab), AGEN 1884 from Agenus, and anti-CTLA4 antibody clone BNI3 from Abcam;

LAG3 modulators such as anti-Lag-3 antibody clone eBioC9B7W (C9B7W) from eBioscience, anti-Lag3 antibody LS-B2237 from LifeSpan Biosciences, IMP701 and LAG525 from Novartis Pharmaceuticals, IMP321 (ImmuFact) from Immutep, anti-Lag3 antibody BMS-986016, BMS-986016 from Bristol-Myers Squibb, REGN3767 from Regeneron Pharmaceuticals, and the LAG-3 chimeric antibody A9H12;

B7-H3 modulators such as MGA271;

KIR modulators such as Lirilumab (IPH2101) from Bristol-Myers Squibb;

CD137 modulators such as urelumab (BMS-663513, Bristol-Myers Squibb), PF-05082566 (anti-4-1BB, PF-2566, Pfizer), or XmAb-5592 (Xencor);

PS modulators such as Bavituximab;

OX40 modulators such as BMS-986178 from Bristol-Myers Squibb, GSK3174998 from GlaxoSmithKline, INCAGN1949 from Agenus, MEDI0562 from MedImmune, PF-04518600 from Pfizer, or RG7888 from Genentech;

GITR modulators such as GWN323 from Novartis Pharmaceuticals, INCAGN1876 from Agenus, or TRX518 from Leap Therapeutics;

TIM3 modulators such as MBG453 from Novartis Pharmaceuticals, or TSR-042 from TESARO; [0204] and modulators such as an antibody or fragments (e.g., a monoclonal antibody, a human, humanized, or chimeric antibody) thereof, RNAi molecules, or small molecules to CD52, CD30, CD20, CD33, CD27, ICOS, BTLA (CD272), CD160, 2B4, LAIR1, TIGHT, LIGHT, DR3, CD226, CD2, or SLAM.

In some embodiments, the additional therapeutic agent comprises a cytokine therapy. Exemplary cytokine drugs include interferon gamma 1-b (Actimmune) from Horizon Pharma; IL-2 based recombinant fusion protein (ALKS 4230) from Alkermes; ALT-801 or ALT-803 from Altor BioScience; AM0010 from ARMO Biosciences; APN301 from Apeiron Biologics; CDX-301/CDX-1401 from Celldex; cergutuzumab amunaleukin (RG7813) or RG7461 from Genentch; CYT-6091 from Cytimmune Sciences; DI-Leu16-IL2 from Alopexx Oncology; GEN-1 from Celsion; heterodimeric IL-15 from Admune Therpeutics; HL143 from HanAll Biopharma; IGN002 from ImmunGene; ImmunoPulse IL-12 from OncoSec Medical; IRX-2 from IRX Therapeutics; M9241 (NHS-IL12) from EMD Serono; MDNA55 from Medicenna Therapeutics; NGR-hTNF from MolMed; or rSIFN-co from Sichuan Huiyang Life Science.

In some embodiments, the additional therapeutic agent comprises an adoptive cell therapy. Exemplary adoptive cell therapies include AFP TCR, MAGE-A10 TCR, or NY-ESO-TCR from Adaptimmune; ACTR087/rituximab from Unum Therapeutics; anti-BCMA CAR-T cell therapy, anti-CD19 “armored” CAR-T cell therapy, JCAR014, JCAR018, JCAR020, JCAR023, JCAR024, or JTCR016 from Juno Therapeutics; JCAR017 from Celgene/Juno Therapeutics; anti-CD19 CAR-T cell therapy from Intrexon; anti-CD19 CAR-T cell therapy, axicabtagene ciloleucel, KITE-718, KITE-439, or NY-ESO-1 T-cell receptor therapy from Kite Pharma; anti-CEA CAR-T therapy from Sorrento Therapeutics; anti-PSMA CAR-T cell therapy from TNK Therapeutics/Sorrento Therapeutics; ATA520 from Atara Biotherapeutics; AU101 and AU105 from Aurora BioPharma; baltaleucel-T (CMD-003) from Cell Medica; bb2121 from bluebird bio; BPX-501, BPX-601, or BPX-701 from Bellicum Pharmaceuticals; BSKO1 from Kiromic; IMCgp100 from Immunocore; JTX-2011 from Jounce Therapeutics; LN-144 or LN-145 from Lion Biotechnologies; MB-101 or MB-102 from Mustang Bio; NKR-2 from Celyad; PNK-007 from Celgene; tisagenlecleucel-T from Novartis Pharmaceuticals; or TT12 from Tessa Therapeutics.

In some embodiments, the additional therapeutic agent comprises an oncolytic viral therapy. Exemplary oncolytic viral therapy include Ad-VirRx 007 from Multivir; CG0070 from Cold Genesys; coxsackievirus (CVA21) from Viralytics; DNX-2401 from DNAtrix; enadenotucirev from PsiOxus Therapeutics; GL-ONC1 from Genelux; HF10 from Takara Bio USA; Imlygic from Amgen; LOAd703 from Lokon Pharma; ONCOS-102 from Targovax; pexastimogene devacirepvec (JX-594) from SilaJen Biotherapeutics; Reolysin from Oncolytics Biotech; telomelysin from Oncolys Biopharma; VSV-INFβ-NIS from Vyriad; adagloxad simolenin from OBI Pharma; ADXS-HER2 and ADXS-PSA from Advaxis; AE37 or AE37/GP2 peptide vaccine from Antigen Express; AlloStim from Immunovative Therapies; AST-VAC1 from Asterias Biotherapeutics; AutoSynVax from Agenus; AVX 701 or AVX 901 from AlphaVax; axalimogene filolisbac (AXAL) from Advaxis; BB-MPI-03 from Benovus Bio; BI 1361849 from Boehringer ingelheim pharmaceuticals; BriaVax from BriaCell Therapeutics; CMB305 from Immune Design; CRS-207 from Aduro Biotech/Incyte; DPX E7 from Immunovaccine; DSP-7888 from Sunovio Pharmaceuticals; GI-6207 or GI-6301 from Celgene; GRN 1201 from GreenPeptide; GL 0817 from Gliknik; IMT1012 from Immunotope; INO-1400, INO-3106, INO-3112, INO-5150, or VGX-3100 from Inovio Pharmaceuticals; MVA p53 vaccine from Tara immune-Oncology; NEO-PV-01 from Bristol-Myers Squibb; PF-06753512 from Pfizer; PVX-410 from OncoPep; SL-701 from Stemline Therapeutics; or WT2725 from Sunovion Pharmaceuticals.

In some instances, the additional therapeutic agent comprises a tyrosine kinase inhibitor. Exemplary tyrosine kinase inhibitors include 706 and AMNI07 (nilotinib). RADOOI, PKC412, gefitinib (IRESSA™), erlotinib (TARCEVA®), sorafenib (EXAVAR®), pazopanib (VOTRIENT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™), vandetanib (ZACTIMA™), and vatalanib.

In some embodiments, the additional therapeutic agent comprises a proteasome inhibitor. Exemplary proteasome inhibitors include bortezomib, carfilzomib, delanzomib, ixazomib, marizomib, oprozomib, or derivatives or analogs thereof.

In some embodiments, the additional therapeutic agent comprises an HDAC inhibitor. Exemplary HDAC inhibitors include ACY-1215 (Rocilinostat), Apicidin, CI-994 (Tacedinaline), CR-2408, entinostat (SNDX-275 or MS-275), ITF2357 (Gavinostat), KD5170, JNJ-26481585, LBH589 (Panobinostat), NVP-LAQ824 (Dacinostat), PXD101 (Belinostat), romidepsin, phenyl butyrate (S-HDAC-42, AR-42), RAS2410 (Resminostat), sodium butyrate, suberoylanilide bis-hydroxamic acid (SBHA), trichostatin-A (TSA), tubacin, valproic acid (VPA), or vorinostat (SAHA).

In some embodiments, the therapeutic agent comprises an encapsulation system, such as a viral capsid, a liposome, or micelle that contains a therapeutic composition such as a drug, a nucleic acid (e.g. an antisense nucleic acid or another nucleic acid to be delivered to the cell), or another therapeutic moiety that is preferably shielded from direct exposure to the circulatory system. Means of preparing liposomes attached to antibodies are well known to those of skill in the art (see, e.g., U.S. Pat. No. 4,957,735, Connor et al. (1985) Pharm. Ther., 28: 341-365, and the like).

### Ischemia-Reperfusion Injury (IRI)

In some embodiments, the disease or condition is an ischemia-reperfusion injury. Following periods of tissue ischemia, the initiation of blood flow causes damage referred to as “ischemia-reperfusion injury” or IRI. IRI contributes to poor outcomes in surgical procedures where IRI occurs due to the necessity to stop blood flow for a period of time and subsequent restoration by therapeutic intervention (e.g., during organ transplantation, cardio/pulmonary bypass procedures, reattachment of severed body parts, reconstructive and cosmetic surgeries and other situations involving stopping and restarting blood flow). Ischemia, or hypoxia, causes physiological changes leading to cell and tissue necrosis and death. Reperfusion induces reactive oxygen species generation, thrombosis, inflammation and cytokine mediated damage.

In some instances, an anti-CD47 antibody is administered to a subject in need thereof for the treatment of an ischemia-reperfusion injury. In some instances, the anti-CD47 antibody is administered to the subject for the treatment of IRI associated with an organ transplantation, skin grafting, surgical resections or tissue reconstruction, reattachment of body parts, treatment of traumatic injury, pulmonary hypertension, sickle cell disease (crisis), myocardial infarction, stroke, surgically-induced ischemia, acute kidney disease/kidney failure, or a condition in which IRI occurs and contributes to the pathogenesis of IRI disease. In some cases, the anti-CD47 antibody inhibits or disrupts the interaction of CD47 and TSP-1.

### Inflammatory Diseases

In some embodiments, the disease or condition is an inflammatory disease. Exemplary inflammatory diseases include, but are not limited to, arthritis, multiple sclerosis, psoriasis, Crohn's disease, inflammatory bowel disease, lupus, Grave's disease and Hashimoto's thyroiditis, and ankylosing spondylitis.

In some instances, an anti-CD47 antibody is administered to a subject in need thereof for the treatment of an inflammatory disease. In some cases, the anti-CD47 antibody is administered to the subject for the treatment of arthritis, multiple sclerosis, psoriasis, Crohn's disease, inflammatory bowel disease, lupus, Grave's disease and Hashimoto's thyroiditis, or ankylosing spondylitis.

### Pathogenic Infections

In some embodiments, the disease or condition is a pathogenic infection. In some cases, described herein is a method of treating a pathogenic infection in a subject in need thereof, which comprises administering to the subject an anti-CD47 antibody described herein.

In some embodiments, the pathogenic infection is a viral infection. Pathogenic viruses include DNA viruses such as single-stranded (ss) DNA viruses, double-stranded (ds) DNA viruses, or DNA viruses that contain both ss and ds DNA regions; and RNA viruses such as single-stranded (ss) RNA viruses or double-stranded (ds) RNA viruses. In some cases, the ssRNA viruses are further classified into positive-sense RNA viruses or negative-sense RNA viruses.

Exemplary dsDNA viruses include viruses from the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

Exemplary ssDNA viruses include viruses from the family: Anelloviridae, Bacillariodnaviridae, Bidnaviridae, Circoviridae, Geminiviridae, Inoviridae, Microviridae, Nanoviridae, Parvoviridae, and Spiraviridae.

Exemplary DNA viruses that contain both ss and ds DNA regions include viruses from the group of pleolipoviruses. In some cases, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

Exemplary dsRNA viruses include viruses from the family: Birnaviridae, Chrysoviridae, Cystoviridae, Endornaviridae, Hypoviridae, Megavirnaviridae, Partitiviridae, Picobirnaviridae, Reoviridae, Rotavirus, and Totiviridae.

Exemplary positive-sense ssRNA viruses include viruses from the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Retroviridae, Secoviridae, Togaviridae, Tombusviridae, Tymoviridae, and Virgaviridae.

Exemplary negative-sense ssRNA viruses include viruses from the family: Arenaviridae, Bornaviridae, Bunyaviridae, Filoviridae, Nyamiviridae, Ophioviridae, Orthomyxoviridae, Paramyxoviridae, and Rhabdoviridae.

In some embodiments, the pathogenic infection is caused by Abelson leukemia virus, Abelson inurine leukemia virus, Abelson's virus, Acute laryngotracheobronchitis virus, Adelaide River virus, Adeno associated virus group, Adenovirus, African horse sickness virus. African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammiillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group. Bunyamwera virus supergroup, Bunyavirus, Burkitt's lymphorna virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LD-H), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis) virus, hepatitis delta virus, hepatitis E virus, hepatitis F virus, hepatitis G virus, hepatitis nonA nonB virus, hepatitis virus, hepatitis virus (nonhuman), bepatoencephalomvelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immodeficiency virus, human imnmodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lynmphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, Jumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytornegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, rnyxoma virus, Myxovirus, Myxovirus multiforrne, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipab virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyarn virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Poly omavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gisbu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, or the Yug Bogdanovac virus.

In some embodiments, the pathogenic infection is caused by a retrovirus. Exemplary retroviruses include, but are not limited to, human immunodefiency virus (HIV), human T-cell leukemia viruses (HTLV), moloney murine leukemia virus (MuLV), murine mammary tumor virus (MMTV), avian leucosis and sarcoma viruses, or Mason-Pfizer monkey virus. In some instances, the anti-CD47 antibody is administered in combination with an antiretroviral therapy.

In some embodiments, the retrovirus is HIV. In some instances, the anti-CD47 antibody is administered in combination with a HIV antiretroviral therapy. Exemplary HIV antiretroviral therapy includes:

nucleoside reverse transcriptase inhibitors (NRTIs) such as abacavir, emtricitabine, lamivudine, tenofovir disoproxil fumarate, and zidovudine;

non-nucleoside reverse transcriptase inhibitors (NNRTIs) such as efavirenz, etravirine, nevirapine, or rilpivirine;

protease inhibitors (PIs) such as atazanavir, darunavir, fosamprenavir, ritonavir, saquinavir, and tipranavir;

fusion inhibitors such as enfuvirtide;

CCR5 antagonists such as maraviroc;

integrase inhibitors such as dolutegravir and raltegravir;

post-attachment inhibitors such as ibalizumab;

pharmacokinetic enhancers such as cobicistat; and

cocktails such as abacavir and lamivudine; abacavir, dolutegravir, and lamivudine;

abacavir, lamivudine, and zidovudine; atazanavir and cobicistat; bictegravir, emtricitabine, and tenofovir alafenamide; darunavir and cobicistat; dolutegravir and rilpivirine; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide fumarate; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; and lopinavir and ritonavir.

In some embodiments, the virus is a hepatitis virus, e.g., hepatitis A, B, C, D, or E. In some instances, the anti-CD47 antibody is administered in combination with an antiviral therapy for hepatitis. Exemplary antiviral therapy for hepatitis include ribavirin; NS3/4A protease inhibitors such as paritaprevir, simeprevir, and grazoprevir; NS5A protease inhibitors such as ledipasvir, ombitasvir, elbasvir, and daclatasvir; NS5B nucleotide/nucleoside and nonnucleoside polymerase inhibitors such as sofosbuvir and dasabuvir; and combinations such as ledipasvir-sofosbuvir, dasabuvir-ombitasvir-paritaprevir-ritonavir; elbasvir-grazoprevir, ombitasvir-paritaprevir-ritonavir, sofosbuvir-velpatasvir, sofosbuvir-velpatasvir-voxilaprevir, and glecaprevir-pibrentasvir; and interferons such as peginterferon alfa-2a, peginterferon alfa-2b, and interferon alfa-2b.

In some embodiments, additional antivirals include acyclovir, brivudin, cidofovir, famciclovir, fomivirsen, foscarnet, ganciclovir, penciclovir, valacyclovir, valganciclovir, or vidarabine.

In some embodiments, the pathogenic infection is a bacterial infection. Exemplary pathogenic bacteria include Gram-positive bacteria from the genus of Actinomyces (e.g., Actinomyces israelii), Bacillus (e.g., Bacillus anthracis, or Bacillus cereus), Clostridium (e.g., Clostridium botulinum, Clostridium difficile, Clostridium pefringens, or Clostridium tetani), Corynebacterium (e.g., Corynebacterium diphtheria), Enterococcus (e.g., Enterococcus faecalis or Enterococcus faecium), Listeria (e.g., Listeria monocytogenes), Staphylococcus (e.g., Staphylococcus aureus, Staphylococcus epidermidis, or Staphylococcus saprophyticus), or Streptococcus (e.g., Streptococcus agalactiae, Streptococcus pneumoniae, or Streptococcus pyogenes); and Gram-negative bacteria from the genus of Bacteroide (e.g., Bacteroides fragilis), Bartonella (e.g., Bartonella henselae or Bartonella Quintana), Bordetella (e.g., Bordetella pertussis), Borrelia (e.g., Borrelia burgdorferi, Borrelia garinii, Borrelia afzelii, or Borrelia recurrentis), Brucella (e.g., Brucella abortus, Brucella canis, Brucella melitensis, or Brucella suis), Campylobacter (e.g., Campylobacter jejuni), Escherichia (e.g., Escherichia coli), Francisella (e.g., Francisella tularensis), Haemophilus (e.g., Haemophilus influenza), Helicobacter (e.g., Helicobacter pylori), Klebsiella (e.g., Klebsiella pneumoniae), Legionella (e.g., Legionella pneumophila), Leptospira (e.g., Leptospira interrogans, Leptospira santarosai, Leptospira weilii, or Leptospira noguchii), Neisseria (e.g., Neisseria gonorrhoeae or Neisseria meningitidis), Pseudomonas (e.g., Pseudomonas aeruginosa), Rickettsia (e.g., Rickettsia rickettsii), Salmonella (e.g., Salmonella typhi or Salmonella typhimurium), Shigella (e.g., Shigella sonnei), Treponema (e.g., Treponema pallidum), Vibrio (e.g., Vibrio cholerae), or Yersinia (e.g., Yersinia pestis, Yersinia enterocolitica, or Yersinia pseudotuberculosis).

In some instances, exemplary pathogenic bacteria include Chlamydia (e.g., Chlamydia pneumoniae or Chlamydia trachomatis), Chlamydophila (e.g., Chlamydophila psittaci), Mycobacterium (e.g., Mycobacterium leprae, Mycobacterium tuberculosis, or Mycobacterium ulcerans), Mycoplasma (e.g., Mycoplasma pneumoniae), or Ureaplasma (e.g., Ureaplasma urealyticum).

In some embodiments, the anti-CD47 antibody is administered in combination with an antibiotic. Exemplary antibiotics include, but are not limited to, penicillins such as penicillin G, penicillin V, methicillin, oxacillin, carbenicillin, nafcillin, or ampicillin; penicillins in combination with β-lactamase inhibitors such as cephalosporins, e.g. cefaclor, cefazolin, cefuroxime, or moxalactam; aminoglycosides such as amikacin, gentamicin, kanamycin, paromomycin, or tobramycin; carbapenems such as cilastatin, doripenem, ertapenem, or meropenem; cloramphenical; lincomycins such as clindamycin or lincomycin; macrolides such as azithromycin, clarithromycin, erythromycin, or fidaxomicin; metronidazole; monobactams; polymyxins; quinolones such as cinoxacin, ciprofloxacin, delafloxacin, gemifloxacin, levofloxacin, lomefloxacin, moxifloxacin, norfloxacin, ofloxacin, sparfloxacin, or trovafloxacin; spectinomycin; sulfonamides such as sulfamethoxazole or sulfisoxazole; tetracyclines such as demeclocycline, doxycycline, minocycline, or oxytetracycline; trimethoprim; or vancomycin.

In some embodiments, the pathogenic infection is an infection from a protozoan. Exemplary parasitic organisms include those from Amoebozoa, Excavata, or Chromalveolata. In some instances, parasitic organisms include Entamoeba histolytica, Plasmodium, Giardia lamblia, and Tryanosoma brucei.

In some embodiments, the anti-CD47 antibody is administered in combination with a drug for the treatment of an antiprotozoal agent. Exemplary antiprotozoal agents include atovaquone, chloroquine, hydroxychloroquine, mefloquine, metronidazole, and pyrimethamine.

### Pharmaceutical Formulation

In some embodiments, the pharmaceutical formulations described herein are administered to a subject by multiple administration routes, including but not limited to, parenteral (e.g., intravenous, intra-arterial, subcutaneous, intramuscular, intraocular, intraperitoneal, intrathecal, intravesical, or intravitreal), oral, intranasal, buccal, rectal, or transdermal administration routes. In some instances, the pharmaceutical composition describe herein is formulated for parenteral (e.g., intravenous, intra-arterial, subcutaneous, intramuscular, intraocular, intraperitoneal, intrathecal, intravesical, or intravitreal) administration. In other instances, the pharmaceutical composition describe herein is formulated for oral administration. In still other instances, the pharmaceutical composition describe herein is formulated for intranasal administration.

In some instances, the pharmaceutical formulations further include pH-adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like.

Stabilizers include compounds such as any antioxidation agents, buffers, acids, preservatives and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic© (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are coadministered. In some instances, the two or more different pharmaceutical compositions are coadministered simultaneously. In some cases, the two or more different pharmaceutical compositions are coadministered sequentially without a gap of time between administrations. In other cases, the two or more different pharmaceutical compositions are coadministered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are optionally reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more anti-CD47 antibodies described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and optionally intended mode of administration and treatment.

For example, the container(s) include an anti-CD47 antibody described herein or an anti-CD47 antibody construct. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, an anti-CD47 antibody described herein is presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., within about ±15%, ±10%, or ±5%.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (x) and lambda (k), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Example 1

Humanized antibodies were designed by creating multiple hybrid sequences that fuse select parts of the parental antibody sequence with the human framework sequences. Three humanized heavy chains were designed based on two different human heavy chain acceptor frameworks and three humanized light chains were designed based on two different human light chain acceptor frameworks. The humanized heavy and light chains were then combined to create the fully humanized antibodies illustrated in Table 7.

TABLE 7

| | HC1 (SEQ ID NO: 123) | HC2 (SEQ ID NO: 124) | HC3 (SEQ ID NO: 125) |
|---|---|---|---|
| LC1 | HC1 (SEQ ID NO: 123) + | HC2 (SEQ ID NO: 124) + | HC3 (SEQ ID NO: 125) + |
| (SEQ ID NO: 126) | LC1 (SEQ ID NO: 126) | LC1 (SEQ ID NO: 126) | LC1 (SEQ ID NO: 126) |
| LC2 | HC1 (SEQ ID NO: 123) + | HC2 (SEQ ID NO: 124) + | HC3 (SEQ ID NO: 125) + |
| (SEQ ID NO: 127) | LC2 (SEQ ID NO: 127) | LC2 (SEQ ID NO: 127) | LC2 (SEQ ID NO: 127) |
| LC3 | HC1 (SEQ ID NO: 123) + | HC2 (SEQ ID NO: 124) + | HC3 (SEQ ID NO: 125) + |
| (SEQ ID NO: 128) | LC3 (SEQ ID NO: 128) | LC3 (SEQ ID NO: 128) | LC3 (SEQ ID NO: 128) |

Calculation of Humanness Scores of Humanized Chains

Humanness scores for monoclonal antibodies were calculated based on the method described in Gao, S. H., Huang, K., Tu, H., and Adler, A. S. 2013. Monoclonal antibody humanness score and its applications. BMC Biotechnology, 13:55. The T20 score represents the degree of antibody humanness by analyzing the primary sequences of the variable regions. The T20 scores for the parental and humanized antibodies are illustrated in Table 8. Based on the method, for full length heavy chains, a score of 79 or above is indicative of looking human-like; for full length kappa light chains, a score of 86 or above is indicative of looking human-like. Because the T20 scores for full length variable regions could be significantly influenced by the low humanness of CDR regions which were kept untouched during humanization, T20 scores for the frameworks of humanized antibodies were also calculated. For heavy chain frameworks, a score of 84 or above is indicative of looking human-like; for kappa light chain frameworks, a score of 90 or above is indicative of looking human-like.

TABLE 8

| | Full-length | |
|---|---|---|
| | (Framework + CDR) | Framework Only |
| | Cutoff = 79 | Cutoff = 84 |
| Parental heavy chain | 70 | 73 |
| HC1 | 87 | 91 |
| HC2 | 85 | 88 |
| HC3 | 85 | 90 |
| Parental light chain | 67 | 73 |
| LC1 | 86 | 97 |
| LC2 | 83 | 95 |
| LC3 | 83 | 95 |

Kinetic Measurement of Humanized Antibodies by Octet

The affinity of 9 humanized antibody combinations and the chimeric parental antibody was evaluated by Octet. Multi-concentration kinetic experiments were performed on the Octet HTX system (ForteBio). Anti-human Fc biosensors (ForteBio) were hydrated in sample diluent (0.1% BSA in PBS and 0.02% Tween 20) and preconditioned in pH 1.9 Glycine. Antigen was diluted by a 7 point, 3 fold serial dilution starting at 500 nM with sample diluent. Antibodies were diluted to 2 ug/mL with sample diluent and then immobilized onto anti-human Fc biosensors. After baselines were established for 30 seconds in sample diluent, the biosensors were moved to wells containing serially diluted antigen to measure the association and dissociation. Association was observed for 180 seconds and dissociation was observed for 300 seconds. The binding affinities were characterized by fitting the kinetic sensorgrams to a monovalent binding model (1:1 binding). The result is illustrated in Table 9.

TABLE 9

| Antibody | KD | Kon | Kdis | | |
|---|---|---|---|---|---|
| Name | (M) | (1/Ms) | (1/s) | Full R\hat{ }2 | Rull X\hat{ }2 |
| Chimeric parental | 1.9E−08 | 1.7E+05 | 3.3E−03 | 0.0256 | 0.9889 |
| HC1 + LC1 | 1.5E−08 | 1.6E+05 | 2.5E−03 | 0.0224 | 0.9860 |
| HC1 + LC2 | 2.6E−08 | 1.6E+05 | 4.1E−03 | 0.0124 | 0.9916 |
| HC1 + LC3 | 1.5E−08 | 1.8E+05 | 2.7E−03 | 0.0218 | 0.9888 |
| HC2 + LC1 | 2.2E−08 | 1.6E+05 | 3.5E−03 | 0.0142 | 0.9907 |
| HC2 + LC2 | 1.8E−08 | 1.7E+05 | 3.0E−03 | 0.0281 | 0.9833 |
| HC2 + LC3 | 2.3E−08 | 1.5E+05 | 3.4E−03 | 0.0185 | 0.9888 |
| HC3 + LC1 | 1.2E−08 | 2.2E+05 | 2.5E−03 | 0.0200 | 0.9884 |
| HC3 + LC2 | 2.9E−08 | 1.2E+05 | 3.6E−03 | 0.0150 | 0.9896 |
| HC3 + LC3 | 1.5E−08 | 1.7E+05 | 2.5E−03 | 0.0141 | 0.9912 |

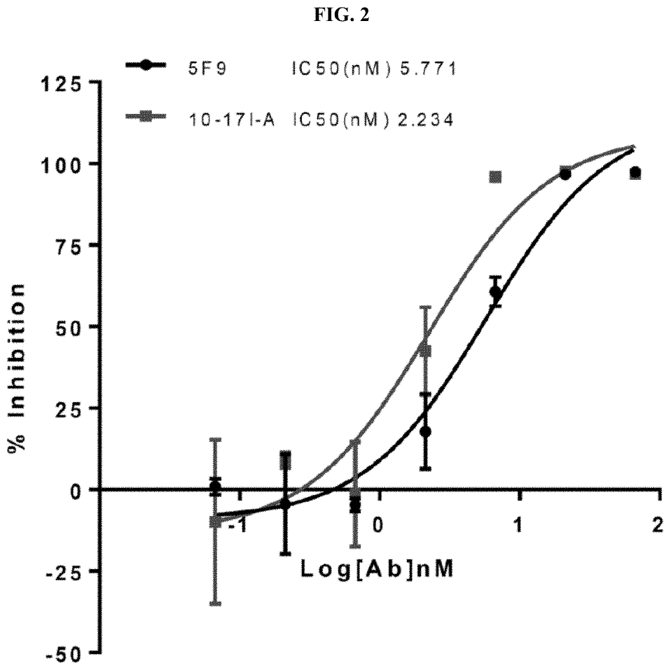

An exemplary humanized antibody (10-17I-A) was tested for blocking of CD47 interaction with SIRPα. FIG. 2 shows the percentage if inhibition in comparison with a reference antibody 5F9 (Forty Seven, Inc.).

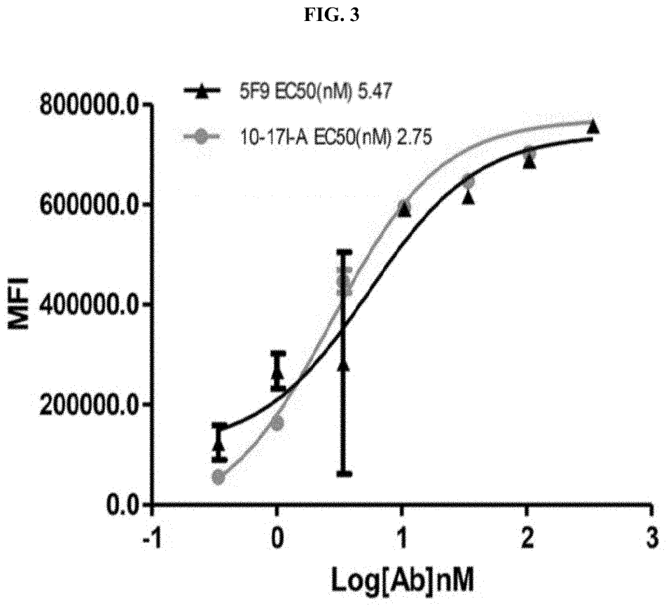

FIG. 3 shows the mean fluorescence intensity (MFI) of 10-17I-A in comparison to a reference antibody 5F9.

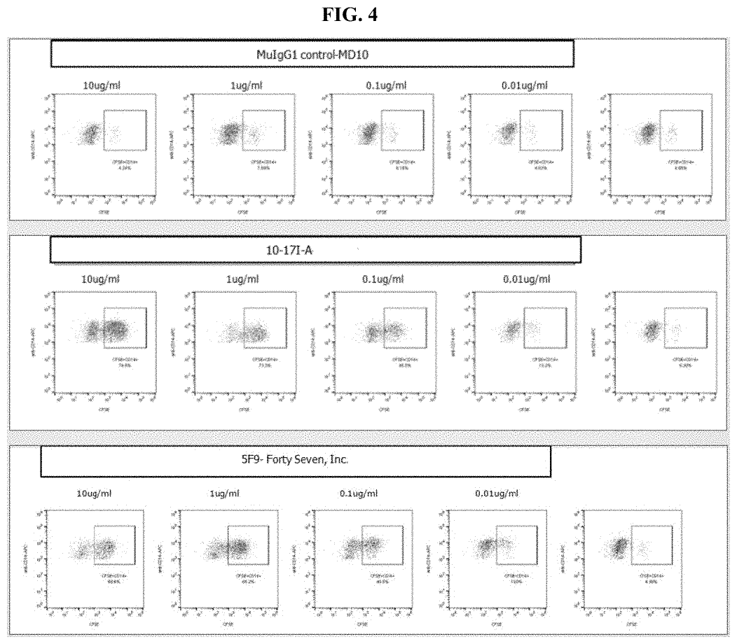

FIG. 4 shows the pyagocytosis of Raji cells in the presence of an IgG1 control antibody, 10-17I-A, and reference antibody 5F9.

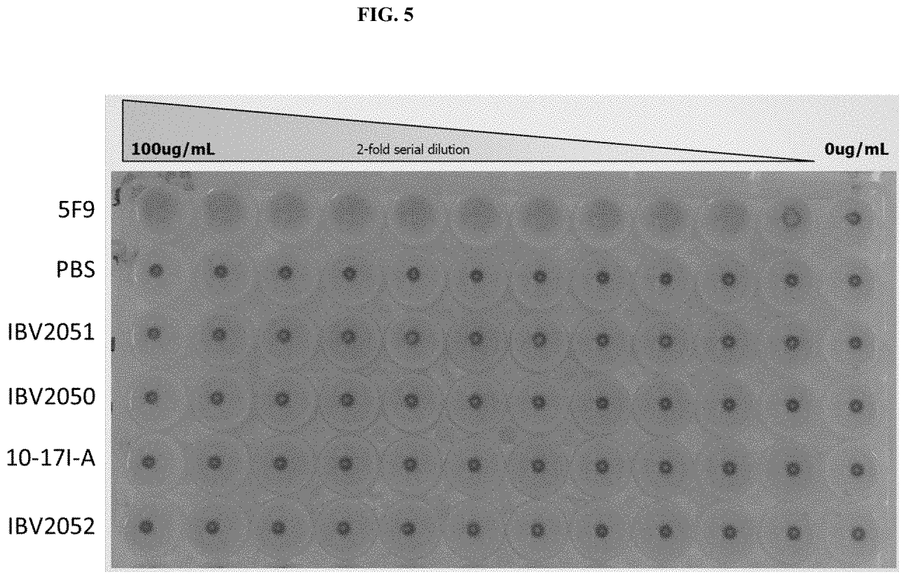

FIG. 5 shows a lack of hemagglutination of exemplary anti-CD47 antibodies described herein and reference antibody 5F9.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An anti-CD47 antibody comprising a variable heavy chain (VH) region and a variable light chain (VL) region, wherein the VH region comprises:
CDR1 sequence GX₁X₂X₃X₄X₅X₆X₇,
wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L;
CDR2 sequence IX₈X₉X₁₀X₁₁X₁₂X₁₃T,
wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and
CDR3 sequence X₁₄X₁₅X₁₆X₁₇X₁₈X₁₉X₂₀X₂₁X₂₂X₂₃X₂₄X₂₅X₂₆,
wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

2. The anti-CD47 antibody of claim 1, wherein the VH region comprises:
CDR1 sequence GX₁X₂X₃X₄X₅X₆X₇,
wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L;
CDR2 sequence IX₈X₉X₁₀X₁₁X₁₂X₁₃T,
wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and
CDR3 sequence selected from SEQ ID NOs: 3-5, 7, 10, 13, 14, 17, 20, 22, 24, 27, 32, 35, 38, 41, and 44.

3. The anti-CD47 antibody of claim 1, wherein the VH region comprises:
CDR1 sequence GX₁X₂X₃X₄X₅X₆X₇,
wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L;
CDR2 sequence selected from SEQ ID NOs: 2, 9, 12, 16, 19, 21, 26, 28, 29, 31, 34, 37, 40, and 43; and
CDR3 sequence X₁₄X₁₅X₁₆X₁₇X₁₈X₁₉X₂₀X₂₁X₂₂X₂₃X₂₄X₂₅X₂₆,
wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

4. The anti-CD47 antibody of claim 1, wherein the VH region comprises:
CDR1 sequence selected from SEQ ID NOs: 1, 6, 8, 11, 15, 18, 23, 25, 30, 33, 36, 39, and 42;
CDR2 sequence IX₉X₉X₁₀X₁₁X₁₂X₁₃T,
wherein X₈ is selected from D, S, N, or L; X₉ is selected from P, C, T, or N; X₁₀ is selected from E, A, Y, S, N, or G; X₁₁ is selected from N, D, I or S; X₁₂ is selected from G, S, C, or T; and X₁₃ is selected from D, N, A, G, Y, E, or S; and
CDR3 sequence X₁₄X₁₅X₁₆X₁₇X₁₈X₁₉X₂₀X₂₁X₂₂X₂₃X₂₄X₂₅X₂₆,
wherein X₁₄ is selected from N, G, or A; X₁₅ is selected from A, R, or K; X₁₆ is selected from W, G, R, Y, or L; X₁₇ is selected from G, L, D, A, S, Y, or V; X₁₈ is selected from G, E, F, Y, R, L, or T; X₁₉ is selected from S, N, R, D, Y, A, F, L, or G; X₂₀ is selected from S, Y, A, G, M, D, W, R, or V; X₂₁ is either present or absence, if present, is selected from S, P, M, N, Y, G, F, A, or D; X₂₂ is either present or absence, if present, is selected from A, D, Y, or T; X₂₃ is either present or absence, if present, is selected from W, Y, F, V, or D; X₂₄ is either present or absence, if present, is selected from F, D, or Y; X₂₅ is either present or absence, if present, is selected from A or Y; and X₂₆ is either present or absence, if present, is Y.

5. The anti-CD47 antibody of claim 1, wherein the VH region comprises:
CDR1 sequence GX₁X₂X₃X₄X₅X₆X₇,
wherein X₁ is selected from F, Y, or H; X₂ is selected from N, S, T, or A; X₃ is selected from I or F; X₄ is selected from K, T, or S; X₅ is selected from D, G, E, S, or N; X₆ is selected from Y, H, T, or N; and X₇ is selected from Y, W, T, I, or L;
CDR2 sequence selected from SEQ ID NOs: 2, 9, 12, 16, 19, 21, 26, 28, 29, 31, 34, 37, 40, and 43; and
CDR3 sequence selected from SEQ ID NOs: 3-5, 7, 10, 13, 14, 17, 20, 22, 24, 27, 32, 35, 38, 41, and 44.

6. The anti-CD47 antibody of claim 1, wherein the VH region comprises:
CDR1 sequence selected from SEQ ID NOs: 1, 6, 8, 11, 15, 18, 23, 25, 30, 33, 36, 39, and 42;
CDR2 sequence selected from SEQ ID NOs: 2, 9, 12, 16, 19, 21, 26, 28, 29, 31, 34, 37, 40, and 43; and
CDR3 sequence selected from SEQ ID NOs: 3-5, 7, 10, 13, 14, 17, 20, 22, 24, 27, 32, 35, 38, 41, and 44.

7. The anti-CD47 antibody of claim 1, wherein the VL region comprises:
CDR1 sequence selected from SEQ ID NOs: 45, 48, 51, 55, 56, 61, 64, 67, 69, 71, or 74;
CDR2 sequence selected from SEQ ID NOs: 46, 49, 52, 57, 62, 65, 72, or 75; and
CDR3 sequence selected from SEQ ID NOs: 47, 50, 53, 54, 58, 59, 60, 63, 66, 68, 70, 73, or 76.

8. The anti-CD47 antibody of claim 1, wherein the VH region comprises CDR1 sequence selected from SEQ ID NOs: 1, 15, 36, and 39; CDR2 sequence selected from SEQ ID NOs: 2, 16, 37, and 40; and CDR3 sequence selected from SEQ ID NOs: 3, 4, 5, 17, 38, and 41; and the VL region comprises CDR1, CDR2, and CDR3 sequences of SEQ ID NOs: 45-47.

9. The anti-CD47 antibody of claim 1, wherein the VH region comprises CDR1, CDR2, and CDR3 sequences of SEQ ID NOs: 6, 2, and 7; and the VL region comprise CDR1, CDR2, and CDR3 sequences of SEQ ID NOs: 48-50.

10. The anti-CD47 antibody of claim 1, wherein the VH region comprises CDR1, CDR2, and CDR3 sequences of SEQ ID NOs: 23, 21, and 24; and the VL region comprise CDR1, CDR2, and CDR3 sequences of SEQ ID NOs: 51-53.

11. The anti-CD47 antibody of claim 1, wherein the VH region comprises CDR1, CDR2, and CDR3 sequences of SEQ ID NOs: 18, 21, and 22; and the VL region comprise CDR1, CDR2, and CDR3 sequences of SEQ ID NOs: 51, 52, and 54.

12. The anti-CD47 antibody of claim 1, wherein the VH region comprises CDR1, CDR2, and CDR3 sequences of SEQ ID NOs: 18-20; and the VL region comprise CDR1, CDR2, and CDR3 sequences of SEQ ID NOs: 55, 52, and 53.

13. The anti-CD47 antibody of claim 1, wherein the VH region comprises CDR1 sequence selected from SEQ ID NO: 25, CDR2 sequence selected from SEQ ID NOs: 26 and 28, and CDR3 sequence selected from SEQ ID NO: 27, and the VL region comprise CDR1, CDR2, and CDR3 sequences of SEQ ID NOs: 56-58.

14. The anti-CD47 antibody of claim 1, wherein the VH region comprises CDR1, CDR2, and CDR3 sequences of SEQ ID NOs: 25, 28, and 27; and the VL region comprise CDR1, CDR2, and CDR3 sequences of SEQ ID NOs: 56, 57, and 59.

15. The anti-CD47 antibody of claim 1, wherein the VH region comprises CDR1, CDR2, and CDR3 sequences of SEQ ID NOs: 25, 29, and 27; and the VL region comprise CDR1, CDR2, and CDR3 sequences of SEQ ID NOs: 56, 57, and 60.

16. The anti-CD47 antibody of claim 1, wherein the VH region comprises CDR1, CDR2, and CDR3 sequences of SEQ ID NOs: 30-32; and the VL region comprise CDR1, CDR2, and CDR3 sequences of SEQ ID NOs: 61-63.

17. The anti-CD47 antibody of claim 1, wherein the VH region comprises CDR1 sequence selected from SEQ ID NO: 11, CDR2 sequence selected from SEQ ID NO: 12, and CDR3 sequence selected from SEQ ID NOs: 13 and 14, and the VL region comprise CDR1, CDR2, and CDR3 sequences of SEQ ID NOs: 64-66.

18. The anti-CD47 antibody of claim 1, wherein the VH region comprises CDR1, CDR2, and CDR3 sequences of SEQ ID NOs: 8-10; and the VL region comprise CDR1, CDR2, and CDR3 sequences of SEQ ID NOs: 67, 65, and 68.

19. The anti-CD47 antibody of claim 1, wherein the VH region comprises CDR1, CDR2, and CDR3 sequences of SEQ ID NOs: 42-44; and the VL region comprise CDR1, CDR2, and CDR3 sequences of SEQ ID NOs: 69, 52, and 70.

20. The anti-CD47 antibody of claim 1, wherein the VH region comprises CDR1, CDR2, and CDR3 sequences of SEQ ID NOs: 33-35; and the VL region comprise CDR1, CDR2, and CDR3 sequences of SEQ ID NOs: 71-73.

21. The anti-CD47 antibody of claim 1, wherein the VH region comprises CDR1, CDR2, and CDR3 sequences of SEQ ID NOs: 11-13; and the VL region comprise CDR1, CDR2, and CDR3 sequences of SEQ ID NOs: 74-76.

22. The anti-CD47 antibody of claim 1, wherein the VH region comprises a sequence selected from Table 3.

23. The anti-CD47 antibody of claim 1, wherein the VL region comprises a sequence selected from Table 3.

24. The anti-CD47 antibody of claim 1, wherein the antibody is a full-length antibody.

25. The anti-CD47 antibody of claim 1, wherein the antibody is a binding fragment.

26. The anti-CD47 antibody of claim 1, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

27. The anti-CD47 antibody of claim 1, wherein the antibody comprises a humanized antibody or binding fragment thereof, a chimeric antibody or binding fragment thereof, a monoclonal antibody or binding fragment thereof, or a bispecific antibody or binding fragment thereof.

28. The anti-CD47 antibody of claim 1, wherein the antibody comprises an IgG1 framework sequence.

29. The anti-CD47 antibody of claim 1, wherein the antibody comprises an IgG2 framework sequence.

30. The anti-CD47 antibody of claim 1, wherein the antibody comprises an IgG4 framework sequence.

31. The anti-CD47 antibody of claim 1, wherein the antibody further comprises a mutation in the Fc region.

32. The anti-CD47 antibody of claim 31, wherein the mutation is S228P.

33. The anti-CD47 antibody of claim 1, wherein the antibody comprises a HC sequence selected from SEQ ID NO: 123-125.

34. The anti-CD47 antibody of claim 1, wherein the antibody comprises a LC sequence selected from SEQ ID NO: 126-128.

35. The anti-CD47 antibody of claim 1, wherein the antibody further comprises a payload.

36. The anti-CD47 antibody of claim 35, wherein the payload comprises a small molecule, a peptide, or a protein.

37. The anti-CD47 antibody of claim 1, wherein the antibody has a humanization score of greater than 85 in both the HC and LC sequences.

38. The anti-CD47 antibody of claim 1, wherein the antibody increases phagocytosis in a target cell relative to the phagocytic activity by a control antibody to an equivalent target cell.

39. The anti-CD47 antibody of claim 1, wherein the antibody has a decreased hemagglutination potential compared to the control antibody.

40. The anti-CD47 antibody of claim 39, wherein the decrease in hemagglutination potential is about 10%, 20%, 30%, 40%, 50%, 60%, 70%, 80%, 90%, 95%, 99%, or higher.

41. The anti-CD47 antibody of claim 39, wherein the decrease in hemagglutination potential is about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or more.

42. The anti-CD47 antibody of claim 1, wherein the antibody does not induce hemagglutination.

43. The anti-CD47 antibody of claim 38, wherein the control antibody is CC-90002 or hu5F9.

44. A nucleic acid polymer encoding an anti-CD47 antibody of claim 1.

45. A vector comprising a nucleic acid polymer of claim 44.

46. A pharmaceutical composition comprising an anti-CD47 antibody of claim 1; and a pharmaceutically acceptable excipient.

47. The pharmaceutical composition of claim 46, wherein the pharmaceutical composition is formulated for systemic administration.

48. The pharmaceutical composition of claim 46, wherein the pharmaceutical composition is formulated for parenteral administration.

49. A method of inducing phagocytosis of a target cell expressing CD47, comprising:
contacting the target cell with an anti-CD47 antibody of claim 1 for a time sufficient for binding of the antibody to CD47, wherein the anti-CD47 antibody blocks interaction of CD47 with SIRPα expressed on a macrophage, thereby inducing phagocytosis of the target cell.

50. The method of claim 49, wherein the target cell is a cancer cell.

51. The method of claim 50, wherein the cancer cell is from bladder cancer, bone cancer, brain cancer, breast cancer, colorectal cancer, eye cancer, head and neck cancer, kidney cancer, liver cancer, lung cancer, pancreatic cancer, prostate cancer, skin cancer, stomach cancer, thyroid cancer, or uterine cancer.

52. The method of claim 50, wherein the cancer cell is from chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

53. The method of claim 49, wherein the target cell is a pathogen-infected cell.

54. The method of claim 53, wherein the pathogen is a virus, a bacterium, or a protozoan.

55. The method of claim 1, wherein the method is an in vivo method.

56. The method of claim 1, wherein the method is an in vitro or ex vivo method.

57. A method of treating a disease or condition in a subject in need thereof, comprising:
administering to the subject an anti-CD47 antibody of claim 1, thereby treating the disease or condition in the subject.

58. The method of claim 57, wherein the subject has a cancer.

59. The method of claim 58, wherein the cancer is a solid tumor.

60. The method of claim 59, wherein the solid tumor is bladder cancer, bone cancer, brain cancer, breast cancer, colorectal cancer, eye cancer, head and neck cancer, kidney cancer, liver cancer, lung cancer, ovarian cancer, pancreatic cancer, prostate cancer, skin cancer, stomach cancer, thyroid cancer, or uterine cancer.

61. The method of claim 58, wherein the cancer is a hematologic malignancy.

62. The method of claim 61, wherein the hematologic malignancy is a B cell lymphoma.

63. The method of claim 61, wherein the hematologic malignancy is a T cell lymphoma.

64. The method of claim 61, wherein the hematologic malignancy is a Hodgkin's lymphoma.

65. The method of claim 61, wherein the hematologic malignancy is a non-Hodgkin's lymphoma.

66. The method of claim 61, wherein the hematologic malignancy is chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

67. The method of claim 1, wherein the cancer is a metastatic cancer.

68. The method of claim 1, wherein the cancer is a relapsed or refractory cancer.

69. The method of claim 57, wherein the subject has a pathogenic infection.

70. The method of claim 57, wherein the method further comprises administering an additional therapeutic agent.

71. The method of claim 70, wherein the additional therapeutic agent comprises an immune checkpoint modulator.

72. The method of claim 71, wherein the immune checkpoint modulator is a modulator of PD-1, PD-L1, PD-L2, CTLA-4, TIM3, LAG3, B7-H3, KIR, 4-1BB, GITR, PS, CD52, CD30, CD20, CD33, CD27, OX40, ICOS, BTLA (CD272), CD160, 2B4, LAIR1, TIGHT, LIGHT, DR3, CD226, CD2, or SLAM.

73. The method of claim 71, wherein the immune checkpoint inhibitor is Pembrolizumab, Nivolumab, Atezolizumab, Avelumab, Durvalumab, or Ipilimumab.

74. The method of claim 70, wherein the additional therapeutic agent comprises chemotherapeutic agent, immunotherapeutic agent, targeted therapeutic agent, hormone-based therapeutic agent, stem-cell based therapeutic agent, or radiation.

75. The method of claim 70, wherein the additional therapeutic agent comprises a first-line therapeutic agent.

76. The method of claim 1, wherein the additional therapeutic agent and the antibody are administered simultaneously.

77. The method of claim 1, wherein the additional therapeutic agent and the antibody are administered sequentially.

78. The method of claim 77, wherein the additional therapeutic agent is administered prior to the antibody.

79. The method of claim 77, wherein the additional therapeutic agent is administered after administration of the antibody.

80. The method of claim 1, wherein the additional therapeutic agent and the antibody are administered as a separate dosage.

81. The method of claim 1, wherein the subject has undergone surgery.

82. The method of claim 1, wherein the subject is a human.

83. A kit comprising an anti-CD47 antibody of claim 1.


SEQUENCE LISTING (140 sequences)

SEQ ID NO: 1 - protein, 8 aa, Artificial Sequence
  Description of Artificial Sequence Synthetic peptide
GFNIKDYY

SEQ ID NO: 2 - protein, 8 aa, Artificial Sequence
  Description of Artificial Sequence Synthetic peptide
IDPENGDT

SEQ ID NO: 3 - protein, 13 aa, Artificial Sequence
  Description of Artificial Sequence Synthetic peptide
NAWGGSSSAWFAY

SEQ ID NO: 4 - protein, 13 aa, Artificial Sequence
  Description of Artificial Sequence Synthetic peptide
NAWGGNYPAWFAY

SEQ ID NO: 5 - protein, 10 aa, Artificial Sequence
  Description of Artificial Sequence Synthetic peptide
NAWLERAMDY

SEQ ID NO: 6 - protein, 8 aa, Artificial Sequence
  Description of Artificial Sequence Synthetic peptide
GFNIKDHY

SEQ ID NO: 7 - protein, 7 aa, Artificial Sequence
  Description of Artificial Sequence Synthetic peptide
NAGLFDY

SEQ ID NO: 8 - protein, 8 aa, Artificial Sequence
  Description of Artificial Sequence Synthetic peptide
GFNIKDTY

SEQ ID NO: 9 - protein, 8 aa, Artificial Sequence
  Description of Artificial Sequence Synthetic peptide
IDPANGNT

SEQ ID NO: 10 - protein, 10 aa, Artificial Sequence
  Description of Artificial Sequence Synthetic peptide
GRGDYYAMDY

SEQ ID NO: 11 - protein, 8 aa, Artificial Sequence
  Description of Artificial Sequence Synthetic peptide
GYSFTGYY

SEQ ID NO: 12 - protein, 8 aa, Artificial Sequence
  Description of Artificial Sequence Synthetic peptide
ISCYNGAT

SEQ ID NO: 13 - protein, 10 aa, Artificial Sequence
  Description of Artificial Sequence Synthetic peptide
ARRDFYGMDY

SEQ ID NO: 14 - protein, 10 aa, Artificial Sequence
  Description of Artificial Sequence Synthetic peptide
ARRDFYAMDY

SEQ ID NO: 15 - protein, 8 aa, Artificial Sequence
  Description of Artificial Sequence Synthetic peptide
GYTFTDYW

SEQ ID NO: 16 - protein, 8 aa, Artificial Sequence
  Description of Artificial Sequence Synthetic peptide
IDTSDSYT

SEQ ID NO: 17 - protein, 9 aa, Artificial Sequence
  Description of Artificial Sequence Synthetic peptide
ARGDYAMNY

SEQ ID NO: 18 - protein, 8 aa, Artificial Sequence
  Description of Artificial Sequence Synthetic peptide
GYTFTEYT

SEQ ID NO: 19 - protein, 8 aa, Artificial Sequence
  Description of Artificial Sequence Synthetic peptide
INPNIGAT

SEQ ID NO: 20 - protein, 8 aa, Artificial Sequence
  Description of Artificial Sequence Synthetic peptide
ARRAYFDY

SEQ ID NO: 21 - protein, 8 aa, Artificial Sequence
  Description of Artificial Sequence Synthetic peptide
INPNNGGT

SEQ ID NO: 22 - protein, 8 aa, Artificial Sequence
  Description of Artificial Sequence Synthetic peptide
GRRSYFDY

SEQ ID NO: 23 - protein, 8 aa, Artificial Sequence
  Description of Artificial Sequence Synthetic peptide
GYTFTEYI

SEQ ID NO: 24 - protein, 8 aa, Artificial Sequence
  Description of Artificial Sequence Synthetic peptide
ARRAYFDY

SEQ ID NO: 25 - protein, 8 aa, Artificial Sequence
  Description of Artificial Sequence Synthetic peptide
GYTFTSNW

SEQ ID NO: 26 - protein, 8 aa, Artificial Sequence
  Description of Artificial Sequence Synthetic peptide
IDPYDCET

SEQ ID NO: 27 - protein, 12 aa, Artificial Sequence
  Description of Artificial Sequence Synthetic peptide
ARYYRYDGYFDY

SEQ ID NO: 28 - protein, 8 aa, Artificial Sequence
  Description of Artificial Sequence Synthetic peptide
IDPYDSET

SEQ ID NO: 29 - protein, 8 aa, Artificial Sequence
  Description of Artificial Sequence Synthetic peptide
IDPYDTET

SEQ ID NO: 30 - protein, 8 aa, Artificial Sequence
  Description of Artificial Sequence Synthetic peptide
GYTFTSHW

SEQ ID NO: 31 - protein, 8 aa, Artificial Sequence
  Description of Artificial Sequence Synthetic peptide
IDPSDSET

SEQ ID NO: 32 - protein, 10 aa, Artificial Sequence
  Description of Artificial Sequence Synthetic peptide
ARLGRAWFAY

SEQ ID NO: 33 - protein, 8 aa, Artificial Sequence
  Description of Artificial Sequence Synthetic peptide
GYTFSSYW

SEQ ID NO: 34 - protein, 8 aa, Artificial Sequence
  Description of Artificial Sequence Synthetic peptide
ILPGSGST

SEQ ID NO: 35 - protein, 10 aa, Artificial Sequence
  Description of Artificial Sequence Synthetic peptide
ARRGLLRFDV

SEQ ID NO: 36 - protein, 8 aa, Artificial Sequence
  Description of Artificial Sequence Synthetic peptide
GYTFTDYW

SEQ ID NO: 37 - protein, 8 aa, Artificial Sequence
  Description of Artificial Sequence Synthetic peptide
IDTSDSYT

SEQ ID NO: 38 - protein, 11 aa, Artificial Sequence
  Description of Artificial Sequence Synthetic peptide
ARLGTGVATDY

SEQ ID NO: 39 - protein, 8 aa, Artificial Sequence
  Description of Artificial Sequence Synthetic peptide
GHTFTDYW

SEQ ID NO: 40 - protein, 8 aa, Artificial Sequence
  Description of Artificial Sequence Synthetic peptide
IDNSDSYT

SEQ ID NO: 41 - protein, 9 aa, Artificial Sequence
  Description of Artificial Sequence Synthetic peptide
AKGYYAMDY

SEQ ID NO: 42 - protein, 8 aa, Artificial Sequence
  Description of Artificial Sequence Synthetic peptide
GYAFTNYL

SEQ ID NO: 43 - protein, 8 aa, Artificial Sequence
  Description of Artificial Sequence Synthetic peptide
INPGSGAT

SEQ ID NO: 44 - protein, 8 aa, Artificial Sequence
  Description of Artificial Sequence Synthetic peptide
ARRVYFDY

SEQ ID NO: 45 - protein, 11 aa, Artificial Sequence
  Description of Artificial Sequence Synthetic peptide
QSLVHSNGNTY

SEQ ID NO: 46 - protein, 3 aa, Artificial Sequence
  Description of Artificial Sequence Synthetic peptide
KVS

SEQ ID NO: 47 - protein, 9 aa, Artificial Sequence
  Description of Artificial Sequence Synthetic peptide
SQSTHVPWT

SEQ ID NO: 48 - protein, 11 aa, Artificial Sequence
  Description of Artificial Sequence Synthetic peptide
QSLVHSNRNTY

SEQ ID NO: 49 - protein, 3 aa, Artificial Sequence
  Description of Artificial Sequence Synthetic peptide
KVS

SEQ ID NO: 50 - protein, 9 aa, Artificial Sequence
  Description of Artificial Sequence Synthetic peptide
SQSTHVPFT

SEQ ID NO: 51 - protein, 6 aa, Artificial Sequence
  Description of Artificial Sequence Synthetic peptide
QSISNY

SEQ ID NO: 52 - protein, 3 aa, Artificial Sequence
  Description of Artificial Sequence Synthetic peptide
YAS

SEQ ID NO: 53 - protein, 9 aa, Artificial Sequence
  Description of Artificial Sequence Synthetic peptide
QQSNSWPFT

SEQ ID NO: 54 - protein, 9 aa, Artificial Sequence
  Description of Artificial Sequence Synthetic peptide
QQSYSWPFT

SEQ ID NO: 55 - protein, 6 aa, Artificial Sequence
  Description of Artificial Sequence Synthetic peptide
QSIGTS

SEQ ID NO: 56 - protein, 6 aa, Artificial Sequence
  Description of Artificial Sequence Synthetic peptide
EDIYNR

SEQ ID NO: 57 - protein, 3 aa, Artificial Sequence
  Description of Artificial Sequence Synthetic peptide
GAT

SEQ ID NO: 58 - protein, 9 aa, Artificial Sequence
  Description of Artificial Sequence Synthetic peptide
QQYWSTPLT

SEQ ID NO: 59 - protein, 9 aa, Artificial Sequence
  Description of Artificial Sequence Synthetic peptide
HQYWSTPLT

SEQ ID NO: 60 - protein, 9 aa, Artificial Sequence
  Description of Artificial Sequence Synthetic peptide
QQYWSSPLT

SEQ ID NO: 61 - protein, 6 aa, Artificial Sequence
  Description of Artificial Sequence Synthetic peptide
ENVGTY

SEQ ID NO: 62 - protein, 3 aa, Artificial Sequence
  Description of Artificial Sequence Synthetic peptide
GAS

SEQ ID NO: 63 - protein, 9 aa, Artificial Sequence
  Description of Artificial Sequence Synthetic peptide
GQSYNYPYT

SEQ ID NO: 64 - protein, 6 aa, Artificial Sequence
  Description of Artificial Sequence Synthetic peptide
QGISNY

SEQ ID NO: 65 - protein, 3 aa, Artificial Sequence
  Description of Artificial Sequence Synthetic peptide
YTS

SEQ ID NO: 66 - protein, 9 aa, Artificial Sequence
  Description of Artificial Sequence Synthetic peptide
QQYSKLPWT

SEQ ID NO: 67 - protein, 5 aa, Artificial Sequence
  Description of Artificial Sequence Synthetic peptide
SSVNY

SEQ ID NO: 68 - protein, 9 aa, Artificial Sequence
  Description of Artificial Sequence Synthetic peptide
QQFTSSTWT

SEQ ID NO: 69 - protein, 6 aa, Artificial Sequence
  Description of Artificial Sequence Synthetic peptide
QSISDY

SEQ ID NO: 70 - protein, 9 aa, Artificial Sequence
  Description of Artificial Sequence Synthetic peptide
QNGHSFPLT

SEQ ID NO: 71 - protein, 7 aa, Artificial Sequence
  Description of Artificial Sequence Synthetic peptide
SSVSSSY

SEQ ID NO: 72 - protein, 3 aa, Artificial Sequence
  Description of Artificial Sequence Synthetic peptide
STS

SEQ ID NO: 73 - protein, 9 aa, Artificial Sequence
  Description of Artificial Sequence Synthetic peptide
QQYSGYPFT

SEQ ID NO: 74 - protein, 6 aa, Artificial Sequence
  Description of Artificial Sequence Synthetic peptide
GNIHNY

SEQ ID NO: 75 - protein, 3 aa, Artificial Sequence
  Description of Artificial Sequence Synthetic peptide
NAK

SEQ ID NO: 76 - protein, 9 aa, Artificial Sequence
  Description of Artificial Sequence Synthetic peptide
QHFWSTPWT

SEQ ID NO: 77 - protein, 120 aa, Artificial Sequence
  Description of Artificial Sequence Synthetic polypeptide
EVQLQQSGAELVRSGASVKLSCTASGFNIKDYYIHWVKQRPEQGLEWIGWIDPENGDTEYVPKFQGKATMTADTSSNTAYLQLSSLTSEDTAVYYCNAWGGSSSAWFAYWGQGTLVTVSA

SEQ ID NO: 78 - protein, 112 aa, Artificial Sequence
  Description of Artificial Sequence Synthetic polypeptide
DVVMTQTPLSLPVSLGDQASISCRSSQSLVHSNGNTYLHWFLQKPGQSPKLLIYKVSNRFSGVPDRFSGSGSGTDFTLKISRVEAEDLGVYFCSQSTHVPWTFGGGTKLEIK

SEQ ID NO: 79 - protein, 119 aa, Artificial Sequence
  Description of Artificial Sequence Synthetic polypeptide
QVQLQQPGAELVRPGASVKLSCKASGYTFTSNWMNWVKQRPEQGLEWIGRIDPYDSETHYNQKFKDKAILTVDKSSNTAYMQLSSLTSEDSAVYYCARYYRYDGYFDYWGQGTTLTVSS

SEQ ID NO: 80 - protein, 107 aa, Artificial Sequence
  Description of Artificial Sequence Synthetic polypeptide
DIQMTQSSSSFSVSLGDRVTITCKASEDIYNRLAWYQQKPGNAPRLLISGATSFETGVPSRFSGSGSGKDYTLSITSLQTEDVATYYCHQYWSTPLTFGAGTKLELK

SEQ ID NO: 81 - protein, 120 aa, Artificial Sequence
  Description of Artificial Sequence Synthetic polypeptide
EVQLQQSGAELVRSGASVKLSCTASGFNIKDYYMHWVKQRPEQGLEWIGWIDPENGDTENAPKFQGKATMTADTSSNTAYLHLSSLTSEDTAVYYCNAWGGNYPAWFAYWGQGTLVTVSA

SEQ ID NO: 82 - protein, 112 aa, Artificial Sequence
  Description of Artificial Sequence Synthetic polypeptide
DVVMTQTPLSLPVSLGDQASISCRSSQSLVHSNGNTYLHWNLQKPGQSPKLLIYKVSNRFSGVPDRFSGSGSGTDFTLKISRVEAEDLGVYFCSQSTHVPWTFGGGTKLEIK

SEQ ID NO: 83 - protein, 114 aa, Artificial Sequence
  Description of Artificial Sequence Synthetic polypeptide
EVQLQQSGAELVRSGASVKLSCTASGFNIKDHYMHWVKQRPEQGLEWIGWIDPENGDTEYVPKFQGKATMTADTSSNTAYLQLSSLTYEDTAVYYCNAGLFDYWGQGTTLTVSS

SEQ ID NO: 84 - protein, 112 aa, Artificial Sequence
  Description of Artificial Sequence Synthetic polypeptide
DVVMTQSPLSLPVSLGDQASISCRSSQSLVHSNRNTYLHWYLQKPGQSPKLLIYKVSNRFSGVPDRFSGSGSGTDFTLKISRVEAEDLGVYFCSQSTHVPFTFGSGTKLEIK

SEQ ID NO: 85 - protein, 117 aa, Artificial Sequence
  Description of Artificial Sequence Synthetic polypeptide
EVQLQQSGAELVRSGASVKLSCTASGFNIKDYYMHWVKQRPEQGLEWIGWIDPENGDTECAPKFQGKATMTADTSSNTAYLQLSSLTSEDTAVYYCNAWLERAMDYWGQGTSVTVSS

SEQ ID NO: 86 - protein, 112 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLKIK

SEQ ID NO: 87 - protein, 116 aa, Artificial Sequence
  Description of Artificial Sequence Synthetic polypeptide
QVQLQQPGAELVMPGASVKMSCKASGYTFTDYWMHWVKQRPGQGLEWIGAIDTSDSYTRYNQKFKGKATLTVDESSSTAYMQLSSLTSEDSAVYYCARGDYAMNYWGQGTSVTVSS

SEQ ID NO: 88 - protein, 112 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 89 - protein, 116 aa, Artificial Sequence
  Description of Artificial Sequence Synthetic polypeptide
QVQLQQAGAELVMPGASVKMSCKASGHTFTDYWMHWVKQRPGQGLEWIGAIDNSDSYTSYNQKFKDKATLTVDESSSTAHMLLSSLTSEDSAVYYCAKGYYAMDYWGQGTSVTVSS

SEQ ID NO: 90 - protein, 112 aa, Artificial Sequence; identical to SEQ ID NO: 88
  Description of Artificial Sequence Synthetic polypeptide

SEQ ID NO: 91 - protein, 115 aa, Artificial Sequence
  Description of Artificial Sequence Synthetic polypeptide
EVQLQQSGPELVKPGASVKISCKTSGYTFTEYIMHWVKQSHGKSLEWIGGINPNNGGTSYNQKFKGKATLTVDKSSSTAYMELRSLTSEDSAVYYCARRAYFDYWGQGTTLTVSS

SEQ ID NO: 92 - protein, 107 aa, Artificial Sequence
  Description of Artificial Sequence Synthetic polypeptide
DIVLTQSPATLSVTPGDRVSLSCRASQSISNYLHWYQQKSHESPRLLIKYASQSISGIPSRFNGSGSGTDFTLSINSVETEDFGMFFCQQSNSWPFTFGSGTKLEIK

SEQ ID NO: 93 - protein, 115 aa, Artificial Sequence
  Description of Artificial Sequence Synthetic polypeptide
EVQLQQSGPELVKPGASVKISCKTSGYTFTEYTMHWVKQSHGKSLEWIGGINPNNGGTTYNQKFKGKATLTVDKSSSTAYMEFRSLTSEDSAVYYCGRRSYFDYWGQGTTLTVSS

SEQ ID NO: 94 - protein, 107 aa, Artificial Sequence
  Description of Artificial Sequence Synthetic polypeptide
DIVLTQSPATLSVTPGDRVSLSCRASQSISNYLHWYQQKSHESPRLLIKYASQSISGIPSRFSGSGSGTDFTLSINSVETEDFGMYFCQQSYSWPFTFGSGTKLEIK

SEQ ID NO: 95 - protein, 119 aa, Artificial Sequence
  Description of Artificial Sequence Synthetic polypeptide
QVQLQQPGAELVRPGASVKLSCKASGYTFTSNWMNWVKQRPEQGLEWIGRIDPYDCETHYNQKFKDKAILTVDKSSNTAYMQLSSLTSEDSAVYYCARYYRYDGYFDYWGQGTTLTVSS

SEQ ID NO: 96 - protein, 107 aa, Artificial Sequence
  Description of Artificial Sequence Synthetic polypeptide
DIQMTQSSSSFSVSLGDRVTITCKASEDIYNRLAWYQQKPGNAPRLLISGATSLETGVPSRFSGSGSGKDYTLSITSLQTEDVATYYCQQYWSTPLTFGAGTKLELK

SEQ ID NO: 97 - protein, 117 aa, Artificial Sequence
  Description of Artificial Sequence Synthetic polypeptide
EVQLQQSGPELVKTGASVKISCKASGYSFTGYYMHWVKQSHGKSLEWIGYISCYNGATSYNQKFKGKATFTVDTSSSTAYMQFNSLTSEDSAVYYCARRDFYGMDYWGQGTSVTVSS

SEQ ID NO: 98 - protein, 107 aa, Artificial Sequence
  Description of Artificial Sequence Synthetic polypeptide
DIQMTQTTSSLSASLGDRVTISCSASQGISNYLNWYQQKPDGTVKLLIYYTSSLHSGVPSRFSGSGSGTDYSLTISNVEPEDIATYYCQQYSKLPWTFGGGTKLEIK

SEQ ID NO: 99 - protein, 119 aa, Artificial Sequence
  Description of Artificial Sequence Synthetic polypeptide
QVQLQQPGAELVRPGASVKLSCKASGYTFTSNWMNWVKQRPEQGLEWIGRIDPYDTETHYSQKFKAKAILTVDKSSNTAYMQLSSLTSEDSAVYYCARYYRYDGYFDYWGQGTTLTVSS

SEQ ID NO: 100 - protein, 107 aa, Artificial Sequence
  Description of Artificial Sequence Synthetic polypeptide
DIQMTQSSSSFSVSLGDRVTITCKASEDIYNRLAWYQQKPGNAPRLLISGATSLETGVPSRFSGSGSGKDYTLSITSLQTEDVATYYCQQYWSSPLTFGAGTKLELK

SEQ ID NO: 101 - protein, 115 aa, Artificial Sequence
  Description of Artificial Sequence Synthetic polypeptide
EVQLQQSGPELVKPGASVKISCKTSGYTFTEYTIHWVKQSHGKSLEWIGGINPNIGATTYNQKFKGKATLTVDKSSSTAYMELRSLTSEDSAVYYCARRAYFDYWGQGTTLTVSS

SEQ ID NO: 102 - protein, 107 aa, Artificial Sequence
  Description of Artificial Sequence Synthetic polypeptide
DILLTQSPAILSVSPGEGVSFSCRASQSIGTSIHWYQQRTNGSPRLLIKYASESVSGIPSRFSGSGSGTGFTLSINSVESEDIADYYCQQSNSWPFTFGSGTKLEIK

SEQ ID NO: 103 - protein, 117 aa, Artificial Sequence
  Description of Artificial Sequence Synthetic polypeptide
EVQLQQSGPELVKTGASVKISCKASGYSFTGYYMHWVKQSHGKSLEWIGYISCYNGATSYNQKFKGKATFTVDTSSSTAYMQFNSLTSEDSAVYYCARRDFYAMDYWGQGTSVTVSS

SEQ ID NO: 104 - protein, 107 aa, Artificial Sequence
  Description of Artificial Sequence Synthetic polypeptide
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 105 - protein, 119 aa, Artificial Sequence; identical to SEQ ID NO: 79
  Description of Artificial Sequence Synthetic polypeptide

SEQ ID NO: 106 - protein, 107 aa, Artificial Sequence
  Description of Artificial Sequence Synthetic polypeptide
DIQMTQSSSSFSVSLGDRVTITCKASEDIYNRLAWYQQKPGNAPRLLISGATSLETGVPSRFSGSGSGKDHTLSITSLQTEDVATYYCQQYWSTPLTFGAGTKLELK

SEQ ID NO: 107 - protein, 117 aa, Artificial Sequence
  Description of Artificial Sequence Synthetic polypeptide
QVQLQQPGAELVRPGASVKLSCKASGYTFTSHWMNWVKQRPGQGLEWIGMIDPSDSETHYNQMFKDKATLTVDKSSSTAYMQLSSLTSEDSAVYYCARLGRAWFAYWGQGTLVTVSA

SEQ ID NO: 108 - protein, 107 aa, Artificial Sequence
  Description of Artificial Sequence Synthetic polypeptide
NIVMTQSPKSMSMSVGERVTLSCKASENVGTYVSWFQQRPDQSPKLLIYGASNRYTGVPDRFTGSGSATDFTLTISSVQAEDLADYHCGQSYNYPYTFGGGTKLEIK

SEQ ID NO: 109 - protein, 117 aa, Artificial Sequence
  Description of Artificial Sequence Synthetic polypeptide
QVQLQQSGAELMKPGASVKISCKATGYTFSSYWIQWVKQRPGHGLEWIGGILPGSGSTNYNEKFKGKATFTADTSSNTAYMQLSSLTSEDSAVYYCARRGLLRFDVWGAGTTVTVSS

SEQ ID NO: 110 - protein, 108 aa, Artificial Sequence
  Description of Artificial Sequence Synthetic polypeptide
ENVLTQSPAIMSASPGEKVTMTCRASSSVSSSYLHWYQQKSGASPKLWIYSTSNLASGVPARFSGSGSGTSYSLTISSVEAEDAATYYCQQYSGYPFTFGSGTKLEIK

SEQ ID NO: 111 - protein, 118 aa, Artificial Sequence
  Description of Artificial Sequence Synthetic polypeptide
QVQLQQPGAEVVMPGASVKMSCKASGYTFTDYWMHWVKQRPGQGLEWIGAIDTSDSYTSYNQKFKGKATLTVDESSSTAYMQLSSLTSEDSAVYYCARLGTGVATDYWGQGTTLTVSS

SEQ ID NO: 112 - protein, 112 aa, Artificial Sequence; identical to SEQ ID NO: 88
  Description of Artificial Sequence Synthetic polypeptide

SEQ ID NO: 113 - protein, 117 aa, Artificial Sequence
  Description of Artificial Sequence Synthetic polypeptide
DVQLQQSGAELVKPGASVKLSCTASGFNIKDTYMHWVKQRPEQGLEWIGRIDPANGNTKYDPKFQGKATITADTSSNTAYLQLSSLTSEDTAVYYCGRGDYYAMDYWGQGTSVTVSS

SEQ ID NO: 114 - protein, 106 aa, Artificial Sequence
  Description of Artificial Sequence Synthetic polypeptide
ENVLTQSPAIMSASLGEKVTMSCRASSSVNYMYWYQQKSDASPKLWIYYTSNLAPGVPARFSGSGSGNSYSLTISSMEGEDAATYYCQQFTSSTWTFGGGTKLEIK

SEQ ID NO: 115 - protein, 117 aa, Artificial Sequence
  Description of Artificial Sequence Synthetic polypeptide
EVQLQQSGPELVKTGASVKISCKASGYSFTGYYIHWVKQSHGKSLEWIGYISCYNGATSYNQKFKGKATFTVDTSSSTGYMQFNSLTSEDSAVYYCARRDFYGMDYWGQGTSVTVSS

SEQ ID NO: 116 - protein, 107 aa, Artificial Sequence
  Description of Artificial Sequence Synthetic polypeptide
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 117 - protein, 115 aa, Artificial Sequence
  Description of Artificial Sequence Synthetic polypeptide
QVQLVQSGAEVKKPGASVKVSCKASGYAFTNYLIEWVRQAPGQGLEWMGVINPGSGATNYNQKFQGRVTMTADKSISTAYMELSRLRSDDTAVYFCARRVYFDYWGQGTLVTVSS

SEQ ID NO: 118 - protein, 115 aa, Artificial Sequence
  Description of Artificial Sequence Synthetic polypeptide
QVQLVQSGAEVKKPGASVKVSCKASGYAFTNYLIEWVRQAPGQGLEWIGVINPGSGATNYNQKFQGRVTLTADKSISTAYMELSRLRSDDTAVYFCARRVYFDYWGQGTLVTVSS

SEQ ID NO: 119 - protein, 115 aa, Artificial Sequence
  Description of Artificial Sequence Synthetic polypeptide
QVQLVQSGAEVKKPGASVKVSCKASGYAFTNYLIEWVRQAPGQGLEWMGVINPGSGATNYNQKFQGRVTMTADKSTSTVYMELSSLRSEDTAVYFCARRVYFDYWGQGTLVTVSS

SEQ ID NO: 120 - protein, 107 aa, Artificial Sequence
  Description of Artificial Sequence Synthetic polypeptide
EIVLTQSPATLSLSPGERATLSCRASQSISDYLHWYQQKPGQAPRLLIKYASQSITGIPARFSGSGSGTDFTLTISSLEPEDFAVYYCQNGHSFPLTFGQGTKLEIK

SEQ ID NO: 121 - protein, 107 aa, Artificial Sequence
  Description of Artificial Sequence Synthetic polypeptide
EIVMTQSPATLSLSPGERATLSCRASQSISDYLHWYQQKPGQAPRLLIKYASQSISGIPARFSGSGSGSDFTLTISSLEPEDFAVYYCQNGHSFPLTFGQGTKLEIK

SEQ ID NO: 122 - protein, 107 aa, Artificial Sequence
  Description of Artificial Sequence Synthetic polypeptide
DIVMTQSPATLSLSPGERATLSCRASQSISDYLHWYQQKPGQAPRLLIKYASQSISGIPARFSGSGSGTDFTLTISSLEPEDFAVYYCQNGHSFPLTFGQGTRLEIK

SEQ ID NO: 123 - protein, 465 aa, Artificial Sequence
  Description of Artificial Sequence Synthetic polypeptide
MDPKGSLSWRILLFLSLAFELSYGQVQLVQSGAEVKKPGASVKVSCKASGYAFTNYLIEWVRQAPGQGLEWMGVINPGSGATNYNQKFQGRVTMTADKSISTAYMELSRLRSDDTAVYFCARRVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 124 - protein, 465 aa, Artificial Sequence
  Description of Artificial Sequence Synthetic polypeptide
MDPKGSLSWRILLFLSLAFELSYGQVQLVQSGAEVKKPGASVKVSCKASGYAFTNYLIEWVRQAPGQGLEWIGVINPGSGATNYNQKFQGRVTLTADKSISTAYMELSRLRSDDTAVYFCARRVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 125 - protein, 465 aa, Artificial Sequence
  Description of Artificial Sequence Synthetic polypeptide
MDPKGSLSWRILLFLSLAFELSYGQVQLVQSGAEVKKPGASVKVSCKASGYAFTNYLIEWVRQAPGQGLEWMGVINPGSGATNYNQKFQGRVTMTADKSTSTVYMELSSLRSEDTAVYFCARRVYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 126 - protein, 234 aa, Artificial Sequence
  Description of Artificial Sequence Synthetic polypeptide
METDTLLLWVLLLWVPGSTGEIVLTQSPATLSLSPGERATLSCRASQSISDYLHWYQQKPGQAPRLLIKYASQSITGIPARFSGSGSGTDFTLTISSLEPEDFAVYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 234 aa, Artificial Sequence
  Description of Artificial Sequence Synthetic polypeptide
METDTLLLWVLLLWVPGSTGEIVMTQSPATLSLSPGERATLSCRASQSISDYLHWYQQKPGQAPRLLIKYASQSISGIPARFSGSGSGSDFTLTISSLEPEDFAVYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 234 aa, Artificial Sequence
  Description of Artificial Sequence Synthetic polypeptide
METDTLLLWVLLLWVPGSTGDIVMTQSPATLSLSPGERATLSCRASQSISDYLHWYQQKPGQAPRLLIKYASQSISGIPARFSGSGSGTDFTLTISSLEPEDFAVYYCQNGHSFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 8 aa, Artificial Sequence
  Description of Artificial Sequence Synthetic peptide
  MOD_RES (2)..(2): Phe, Tyr, or His
  MOD_RES (3)..(3): Asn, Ser, Thr, or Ala
  MOD_RES (4)..(4): Ile or Phe
  MOD_RES (5)..(5): Lys, Thr, or Ser
  MOD_RES (6)..(6): Asp, Gly, Glu, Ser, or Asn
  MOD_RES (7)..(7): Tyr, His, Thr, or Asn
  MOD_RES (8)..(8): Tyr, Trp, Thr, Ile, or Leu
GXXXXXXX

SEQ ID NO: 130 - protein, 8 aa, Artificial Sequence
  Description of Artificial Sequence Synthetic peptide
  MOD_RES (2)..(2): Asp, Ser, Asn, or Leu
  MOD_RES (3)..(3): Pro, Cys, Thr, or Asn
  MOD_RES (4)..(4): Glu, Ala, Tyr, Ser, Asn, or Gly
  MOD_RES (5)..(5): Asn, Asp, Ile, or Ser
  MOD_RES (6)..(6): Gly, Ser, Cys, or Thr
  MOD_RES (7)..(7): Asp, Asn, Ala, Gly, Tyr, Glu, or Ser
IXXXXXXT

SEQ ID NO: 131 - protein, 13 aa, Artificial Sequence
  Description of Artificial Sequence Synthetic peptide
  MOD_RES (1)..(1): Asn, Gly or Ala
  MOD_RES (2)..(2): Ala, Arg, or Lys
  MOD_RES (3)..(3): Trp, Gly, Arg, Tyr, or Leu
  MOD_RES (4)..(4): Gly, Leu, Asp, Ala, Ser, Tyr, or Val
  MOD_RES (5)..(5): Gly, Glu, Phe, Tyr, Arg, Leu, or Thr
  MOD_RES (6)..(6): Ser, Asn, Arg, Asp, Tyr, Ala, Phe, Leu or Gly
  MOD_RES (7)..(7): Ser, Tyr, Ala, Gly, Met, Asp, Trp, Arg, or Val
  MOD_RES (8)..(8): Ser, Pro, Met, Asn, Tyr, Gly, Phe, Ala, Asp, or not present
  MOD_RES (9)..(9): Ala, Asp, Tyr, Thr, or not present
  MOD_RES (10)..(10): Trp, Tyr, Phe, Val, Asp, or not present
  MOD_RES (11)..(11): Phe, Asp, Tyr, or not present
  MOD_RES (12)..(12): Ala, Tyr, or not present
  MISC_FEATURE (13)..(13): May or may not be present
XXXXXXXXXXXXY

SEQ ID NO: 132 - protein, 8 aa, Artificial Sequence
  Description of Artificial Sequence Synthetic peptide
  MOD_RES (7)..(7): Tyr, His, or Thr
GFNIKDXY

SEQ ID NO: 133 - protein, 8 aa, Artificial Sequence
  Description of Artificial Sequence Synthetic peptide
  MOD_RES (4)..(4): Glu or Ala
  MOD_RES (7)..(7): Asp or Asn
IDPXNGXT

SEQ ID NO: 134 - protein, 13 aa, Artificial Sequence
  Description of Artificial Sequence Synthetic peptide
  MOD_RES (1)..(1): Asn or Gly
  MOD_RES (2)..(2): Ala or Arg
  MOD_RES (3)..(3): Trp or Gly
  MOD_RES (4)..(4): Gly, Leu, or Asp
  MOD_RES (5)..(5): Gly, Glu, Phe, or Tyr
  MOD_RES (6)..(6): Ser, Asn, Arg, Asp, or Tyr
  MOD_RES (7)..(7): Ser, Tyr, or Ala
  MOD_RES (8)..(8): Ser, Pro, Met, or not present
  MOD_RES (9)..(9): Ala, Asp, or not present
  MOD_RES (10)..(10): Trp, Tyr, or not present
  MISC_FEATURE (11)..(11): May or may not be present
  MISC_FEATURE (12)..(12): May or may not be present
  MISC_FEATURE (13)..(13): May or may not be present
XXXXXXXXXXFAY

SEQ ID NO: 135 - protein, 8 aa, Artificial Sequence
  Description of Artificial Sequence Synthetic peptide
  MOD_RES (5)..(5): Thr or Ser
  MOD_RES (6)..(6): Asp, Glu, or Ser
  MOD_RES (7)..(7): Tyr, Asn, or His
  MOD_RES (8)..(8): Trp, Thr, or Ile
GYTFXXXX

SEQ ID NO: 136 - protein, 8 aa, Artificial Sequence
  Description of Artificial Sequence Synthetic peptide
  MOD_RES (2)..(2): Asp, Ser, Asn, or Leu
  MOD_RES (3)..(3): Pro, Cys, or Thr
  MOD_RES (4)..(4): Tyr, Ser, Asn, or Gly
  MOD_RES (5)..(5): Asn, Asp, Ile, or Ser
  MOD_RES (6)..(6): Gly, Ser, Cys, or Thr
  MOD_RES (7)..(7): Ala, Tyr, Gly, Glu, or Ser
IXXXXXXT

SEQ ID NO: 137 - protein, 12 aa, Artificial Sequence
  Description of Artificial Sequence Synthetic peptide
  MOD_RES (1)..(1): Gly or Ala
  MOD_RES (3)..(3): Gly, Arg, Tyr, or Leu
  MOD_RES (4)..(4): Asp, Ala, Ser, Tyr, or Gly
  MOD_RES (5)..(5): Phe, Tyr, Arg, Leu, or Thr
  MOD_RES (6)..(6): Tyr, Ala, Phe, Leu, or Gly
  MOD_RES (7)..(7): Ala, Met, Asp, Trp, Arg, or Val
  MOD_RES (8)..(8): Met, Asn, Tyr, Gly, Phe, or Ala
  MOD_RES (9)..(9): Asp, Tyr, Ala, Thr, or not present
  MOD_RES (10)..(10): Tyr, Phe, Val, Asp, or not present
  MOD_RES (11)..(11): Asp, Tyr, or not present
  MISC_FEATURE (12)..(12): May or may not be present
XRXXXXXXXXXY

SEQ ID NO: 138 - protein, 4 aa, Artificial Sequence
  Description of Artificial Sequence Synthetic peptide
GGFG

SEQ ID NO: 139 - protein, 4 aa, Artificial Sequence
  Description of Artificial Sequence Synthetic peptide
ALAL

SEQ ID NO: 140 - protein, 4 aa, Artificial Sequence
  Description of Artificial Sequence Synthetic peptide
GFLG